(19) United States
(12) Patent Application Publication
(10) Pub. No.: US 2024/0197744 A1
(43) Pub. Date: Jun. 20, 2024

(54) TREATING SEX STEROID DEPENDENT CANCER WITH BMX INHIBITORS

(71) Applicant: The Cleveland Clinic Foundation, Cleveland, OH (US)

(72) Inventor: Nima SHARIFI, Cleveland, OH (US)

(21) Appl. No.: 18/557,011

(22) PCT Filed: May 10, 2022
(86) PCT No.: PCT/US2022/028544

§ 371 Date: Oct. 24, 2023

Related U.S. Application Data
(60) Provisional application No. 63/187,655, filed on May 12, 2021.

(51) Int. Cl. A61K 31/519; A61K 31/4985; A61P 35/00; C07K 16/40; C12Q 1/6886; G01N 33/574

(52) U.S. Cl. CPC A61K 31/519; A61K 31/4985; A61P 35/00; C07K 16/40; C12Q 1/6886; G01N 33/57484; C07K 2317/76; G01N 2333/904

(57) ABSTRACT

The present invention provides methods, kits, and compositions for treating sex steroid dependent cancer using BMX inhibitors. In certain embodiments, a sample from a subject having, or suspected of having, a sex steroid dependent disease cancer, is further assayed to determine: i) if the subject is heterozygous or homozygous for the HSD3B1(1245C) allele that encodes for the 3βSD1(367T) protein. In some embodiments, the BMX inhibitor comprises a monoclonal antibody or BMX binding portion thereof (e.g., zanubrutinib, acalabrutinib, abivertinib or ibrutinib).

The present application claims priority to U.S. Provisional application Ser. No. 63/187,655, filed May 12, 2021, which is herein incorporated by reference in its entirety.

This invention was made with government support under CA261995, CA236780, CA172382, CA249279, CA251961, and RR031537 awarded by the National Institutes of Health and W81XWH2010093, W81XWH2010137 and W81XWH2210082 awarded by the Department of Defense. The government has certain rights in the invention.

### SEQUENCE LISTING

The text of the computer readable sequence listing filed herewith, titled “39530-601_SEQUENCE_LISTING_ST25”, created May 10, 2022, having a file size of 3,393 bytes, is hereby incorporated by reference in its entirety.

### FIELD OF THE INVENTION

The present invention provides methods, kits, and compositions for treating sex steroid dependent cancer (e.g., prostate cancer) using BMX inhibitors. In certain embodiments, a sample from a subject having, or suspected of having, a sex steroid dependent cancer, is further assayed to determine: i) if the subject is heterozygous or homozygous for the HSD3B1(1245C) allele that encodes for the 3βHSD1(367T) protein. In some embodiments, the BMX inhibitor comprises a monoclonal antibody or BMX binding portion thereof (e.g., zanubrutinib, acalabrutinib, abivertinib or ibrutinib).

### BACKGROUND OF THE INVENTION

Prostate cancer is the most common cause of non-skin cancer and second leading cause of cancer death in U.S. men, with approximately 191,000 new cases and 33,000 deaths estimated for 2020¹. Androgen deprivation therapy (ADT), with medical or surgical castration, is the traditional, long-standing frontline treatment for advanced prostate cancer and established the standard of care over 70 years ago². The essential therapeutic maneuver with ADT is depletion of circulating gonadal testosterone, which, however, leaves non-testicular physiologic androgens intact³. With ADT alone, tumor responses occur in 80-90% of treated patients, yet median response durations vary widely, reflecting tumor heterogeneity. In recent years, several large phase 3 clinical trials have demonstrated a profound survival benefit for addition of 1 of 4 agents (abiraterone, docetaxel, enzalutamide or apalutamide)⁴⁻¹⁰ to intensify treatment initiated with ADT for metastatic castration-sensitive prostate cancer (CSPC). Unfortunately, metastatic disease almost always eventually progresses as castration-resistant prostate cancer (CRPC)^{11,12}. Although CRPC is heterogeneous, the regeneration of potent androgens and other mechanisms that stimulate the androgen receptor (AR) are major drivers of resistance, as is evidenced by the survival benefit conferred by blocking androgen synthesis (e.g., CYP17A1 inhibition) or directly blocking AR with potent antagonists¹³⁻¹⁷. However, resistance to these agents, including abiraterone and enzalutamide, eventually occurs and leads to death. Genetic clinical evidence now demonstrates a clear role for the enzyme 3β-hydroxysteroid dehydrogenase-1 (3βHSD1; encoded by HSD3B1) in resistance to multiple hormonal therapies, including abiraterone and enzalutamide.

The vast majority of prostate cancers are dependent on androgen activation of AR. Treatment of metastatic disease with ADT (i.e., castration, or gonadal testosterone depletion) is initially effective in most patients. Testosterone (T) modestly and directly stimulates AR, but the major androgenic activity occurs after conversion by steroid-5α-reductase (SRD5A) isoenzymes to the more potent 5α-dihydrotestosterone (DHT), which is the major androgen found bound to AR in the prostate cell nucleus¹⁸. The ultimate effect of ADT is therefore intratumoral depletion of DHT. Unfortunately, the clinical response to ADT is almost always temporary and clinical studies of CRPC have consistently demonstrated that intratumoral concentrations of DHT are elevated to physiologically relevant levels that stimulate AR¹⁹⁻²¹. The regeneration of DHT occurring with low serum T is due to intratumoral androgen generation from precursors that may originate from de novo steroidogenesis from cholesterol or from tumors that can utilize adrenal precursor steroids^{22,23}.

An overview of the metabolic pathways of androgen synthesis points to the critical role for 3βHSD1. There are at least 3 possible pathways to DHT synthesis from non-gonadal precursors 1) The canonical pathway requires conversion from adrenal dehydroepiandrosterone (DHEA)→androstenedione→testosterone→DHT; 2) the 5α-androstanedione pathway still utilizes adrenal precursors although it circumvents testosterone and converts androstenedione→5α-androstanedione→DHT; 3) the “backdoor” pathways may occur using de novo steroidogenesis from cholesterol, by way of 5α-reduction of progesterone or 17OH-progesterone, which then requires 5α-androstanediol as an intermediate metabolite that is then converted to DHT (not shown). Alternatively, de novo steroidogenesis may go through the testosterone-dependent canonical pathway. Notably, all of these pathways require CYP17A1, which is the pharmacologic target of abiraterone. Evidence exists for all 3 pathways, and our prior work suggests that when tumors utilize adrenal precursors, the 5α-androstanedione pathway is dominant in models as well as in studies of freshly collected metastatic CRPC tissues²⁴. Nonetheless, all pathways for synthesis of testosterone and/or DHT also require 3βHSD enzymatic activity. 3βHSD performs 2 reactions necessary to convert the 3β-OH, Δ⁵-structure of DHEA and cholesterol (FIG. 1) to biologically active androgens: oxidation of 3β-OH to 3-keto and Δ⁵→Δ⁴ isomerization^{25,26}. Humans have 2 isoenzymes for 3βHSD, 3βHSD1 being the predominant peripherally expressed isoenzyme^{26,27}. Together, these observations bring the central role of 3βHSD1 in prostate cancer into focus.

The art previously described a gain-of-function missense in 3βHSD1 (encoded by HSD3B1) that increases DHT synthesis and spurs development of CRPC²⁸. A single nucleotide change that converts A→C at position 1245 of HSD3B1 exchanges an asparagine (N) for a threonine (T) at 3βHSD1 amino acid position 367. This missense may occur as a somatic mutation but perhaps even more important for predictive biomarkers is that it is a frequent germline variation and is present at an allele frequency of approximately 25-35%. The HSD3B1(1245C) allele that encodes for the 3βHSD1(367T) enzyme missense impedes ubiquitination and rapid proteasome-mediated degradation, thereby increasing steady-state 3βHSD1 protein levels. This hastens what is normally the rate-limiting step of DHT synthesis from extragonadal precursors, thus regenerating tumor tissue androgens and promoting the development of CRPC²⁸. The HSD3B1(1245A) allele is the adrenal-restrictive allele because it limits conversion from adrenal DHEA to DHT whereas HSD3B1(1245C) is the adrenal-permissive allele as it enables robust conversion from DHEA to downstream DHT^{29,30}.

Inheritance of the adrenal-permissive HSD3B1(1245C) allele enables robust DHT synthesis from extragonadal (mainly adrenal) precursor steroids and thus after treatment with ADT, is a metabolic driver of more rapid progression to CRPC and disease lethality. We suspected that the major contribution of adrenal androgen with adrenal-permissive and adrenal-restrictive HSD3B1 genetics and physiology would occur after medical castration. We initially tested this hypothesis in 3 cohorts treated with ADT: 118 patients with recurrence after radical prostatectomy treated with ADT at the time of PSA relapse at Cleveland Clinic (cohort 1), a comparable cohort of 137 patients treated with ADT at Mayo Clinic (cohort 2) and 188 men with metastatic prostate cancer treated with ADT at Mayo Clinic (cohort 3)³¹. In all 3 cohorts, patients with HSD3B1(1245C) allele inheritance had significantly worse clinical outcomes after ADT, with the most profound effect evident for men who inherit 2 copies of the HSD3B/(1245C) allele (approximately 10% of men)³¹.

It was further determined that HSD3B1(1245C) allele inheritance drives poor outcomes in men treated with ADT for biochemical recurrence after radiotherapy for localized prostate cancer at Dana-Farber Cancer Institute³². Finally, it was determined that inheritance of a single HSD3B1(1245C) allele (more than half of the White population) drives poor clinical outcomes and shortens overall survival after ADT in low-volume metastatic CSPC in a phase 3 clinical trial of ADT+/−docetaxel³³. Importantly, HSD3B1-driven adverse biology and clinical outcomes are not addressed by taxane therapy because there is no interaction between HSD3B1 genotype and benefit from docetaxel³³. Notably, the discovery of HSD3B1(1245C)-driven adverse cancer outcomes after ADT have been independently validated in other studies, including in cohorts from Spain and Japan³⁴⁻³⁶.

### SUMMARY OF THE INVENTION

The present invention provides methods, kits, and compositions for treating a sex steroid dependent cancer using BMX inhibitors. In certain embodiments, a sample from a subject having, or suspected of having, a sex steroid dependent cancer (e.g., prostate cancer), is further assayed to determine: i) if the subject is heterozygous or homozygous for the HSD3B1(1245C) allele that encodes for the 3βHSD1(367T) protein. In some embodiments, the BMX inhibitor comprises a monoclonal antibody or BMX binding portion thereof (e.g., zanubrutinib, acalabrutinib, abivertinib or ibrutinib).

In some embodiments, provided herein are methods of treating a sex steroid dependent cancer (e.g., prostate cancer) comprising: treating a subject having a sex steroid dependent cancer with a cytoplasmic tyrosine-protein kinase BMX (BMX) inhibitor (e.g., administering said BMX inhibitor to said subject, or providing said BMX inhibitor to said subject such that they administer it to themselves).

In particular embodiments, the sex steroid dependent cancer is prostate cancer. In some embodiments, the prostate cancer comprises castration-resistant prostate cancer (CRPC). In other embodiments the sex steroid dependent cancer is breast cancer, ovarian cancer, or endometrial cancer.

In particular embodiments, the BMX inhibitor comprises an shRNA sequence (e.g., as shown in Table 1, or as generated using software known in the art). In other embodiments, the BMX inhibitor comprises an anti-BMX monoclonal antibody or BMX binding portion thereof (Fab fragment, etc.). In particular embodiments, the BMX inhibitor comprises zanubrutinib. In other embodiments, the BMX inhibitor comprises acalabrutinib, abivertinib, ibrutinib, Elsubrutinib, & ABBV-599 (combo of ABBV-105 BTKi+494 JAKli); abiverinib; Brukinsa zanubrutinib; BIIB091; Vecabrutinib; BMS-986142; spebrutinib; AS-0871; AS-1763, CB-1763; CG-806; DWP213388; LY3527727; fenebrutinib; ICP-022; SHR-1459, TG-1701; HCI-1401; LSK9985; MK-1026, ARQ 531; evobrutinib; M7583 TL-895: Remibrutinib; Nurix-BTK; Tirabrutinib; PRN2246, SAR442168; PRN473 Topical; rilzabrutinib (PRN1008); TP-4207; TAK-020; DTRM-12; and DTRMWXHS-12.

In some embodiments, the subject is 367T in the3βHSD1 protein. In other embodiments, the methods further comprise: conducting an assay on a sample from said subject to determine if the subject is 367T or 367N in their 3βHSD1 protein. In other embodiments, the subject is heterozygous or homozygous for 1245C in the HSD3B1 gene. In other embodiments, the methods further comprise: conducting an assay on a sample from said subject to determine if said subject is heterozygous or homozygous for 1245C in the HSD3B1 gene. In additional embodiments, the subject is a male (e.g., human male) or female (e.g., human female).

In some embodiments, provided herein are in vitro compositions comprising: a) human sex steroid dependent cancer cells (e.g., prostate cancer cells); and b) a BMX inhibitor that is exogenous to said human sex steroid dependent cancer cells. In particular embodiments, the sex steroid dependent cancer cells are breast cancer cells, ovarian cancer cells, or endometrial cancer cells.

In particular embodiments, the BMX inhibitor comprises an shRNA sequence (e.g., as shown in Table 1, or as generated using software known in the art). In other embodiments, the BMX inhibitor comprises an anti-BMX monoclonal antibody or BMX binding portion thereof. In further embodiments, the BMX inhibitor comprises zanubrutinib. In some embodiments, wherein the BMX inhibitor comprises acalabrutinib, Elsubrutinib, & ABBV-599 (combo of ABBV-105 BTKi+494 JAKli); abiverinib; Brukinsa zanubrutinib; BIIB091; Vecabrutinib; BMS-986142; spebrutinib; AS-0871; AS-1763, CB-1763; CG-806; DWP213388; LY3527727; fenebrutinib; ICP-022; SHR-1459, TG-1701; HCI-1401; LSK9985; MK-1026, ARQ 531; evobrutinib; M7583 TL-895; Remibrutinib; Nurix-BTK; Tirabrutinib; PRN2246, SAR442168; PRN473 Topical; rilzabrutinib (PRN1008); TP-4207; TAK-020; DTRM-12; and DTRMWXHS-12.

In particular embodiments, the sex steroid dependent cells are human prostate cancer cells which comprise castration-resistant prostate cancer cells. In further embodiments, the sex steroid dependent cancer cells (e.g., prostate cancer cells) are 367T in the3βHSD1 protein. In additional embodiments, the sex steroid dependent cancer cells (e.g., prostate cancer cells) are heterozygous or homozygous for 1245C in the HSD3B1 gene.

### DESCRIPTION OF THE FIGURES

The patent or application file contains at least one drawing executed in color. Copies of this patent or patent application publication with color drawings will be provided by the Office upon request and payment of the necessary fee.

FIG. 1. 3β-hydroxysteroid dehydrogenase (3βHSD1) pY344 is required for DHEA metabolism. A. C4-2 cells overexpressing HA-3βHSD1 were treated with or without DHEA for 1 hour. Pan-phospho-tyrosine (pTyr) was detected by immunoprecipitation and western blot. B. 3βHSD1-GST was transfected, and cells were treated with DHEA for 1 hour. GST pull-down complexes were immunoblotted, and the indicated phosphopeptides on human 3βHSD1 were identified by LC-MS/MS. A doubly charged peptide with a mass of 896.91 Da was identified in the survey analysis of GST-HSD3B1. The CID spectra for this peptide are dominated by singly charged C-terminal y ions. The mass difference between y₇ and y₆ is consistent with modification at Y344. C. Cells were transfected with HA-3βHSD1 mutants and treated as in (A). D. 3βHSD1 enzyme activity was assessed by analyzing DHEA metabolism by HPLC. Cells were transfected with Flag-3βHSD1 mutants and subsequently treated with [³H]-DHEA for 4 hours, followed by steroid extraction from media, steroid separation, and quantitation with HPLC. The experiment was done in triplicate and repeated in independent experiments. Shown are the steroid sites of 3βHSD1 biochemical modification. E. C4-2 cells overexpressing 3βHSD1-GST were treated with ethanol or DHEA, pregnenolone, or androstenediol for 1 hour. GST pull-down complexes were immunoblotted with a phospho-3βHSD1-Y344 antibody. For all panels, unless otherwise noted, error bars represent the SEM; P values were calculated using un-paired two-tailed t tests. * P<0.05.

FIG. 2. BMX is required for DHEA metabolism by 3βHSD1. A. LNCaP cells were treated with ibrutinib or zanubrutinib for 1 hour and subsequently treated with [³H]-DHEA for 5 hours, followed by steroid extraction from media and steroid separation and quantitation with HPLC. The experiment was done in triplicate and repeated in independent experiments. B. Cells stably expressing shNT or two shRNA sequences against BMX were treated with [³H]-DHEA for 6 hours and analyzed as in (A). C. 293T cells were transiently co-transfected with HA-BMX, EGFR, SRC, or YES and GST-3βHSD1, followed by GST pull-down and western blot. D. 293T cells were transiently co-transfected with HA-BMX and GST-3βHSD1, followed by HA immunoprecipitation and western blot. E. LNCaP cells were transiently co-transfected with HA-BMX and GST-3βHSD1. Cells were starved with medium containing 10% charcoal-stripped fetal bovine serum for 24 hours and then treated with steroids for 2 hours, followed by GST-pull down and western blot to detect interaction of HA-BMX and GST-3βHSD1. F. LNCaP cells were starved with medium containing 10% charcoal-stripped fetal bovine serum for 24 hours and then transfected with HA-BMX and treated with steroids for 2 hours; p-BMX was detected by western blot. G. Stable C4-2 cell lines with HSD3B1 gRNA or control gRNA were transfected with HA-BMX and starved with medium containing 10% charcoal-stripped fetal bovine serum for 24 hours and then treated with DHEA for 2 hours; p-BMX was detected by western blot. For all panels, unless otherwise noted, error bars represent the SEM; P values were calculated using un-paired two-tailed t tests. * P<0.05. ** P<0.01.*** P<0.001.

FIG. 3. BMX directly binds 3βHSD1 and phosphorylates Y344. A. LNCaP cells overexpressing 3βHSD1 were treated with ibrutinib for 1.5 hours with or without DHEA for 0.5 hours. Pan-phospho-tyrosine (pTyr) was detected by immunoprecipitation and western blot. B and C. Cells overexpressing 3βHSD1 were treated with ibrutinib or zanubrutinib for 3 hours, and DHEA for 1 or 2 hours, respectively. Phospho-3βHSD1-Y344 was detected by immunoprecipitation and western blot. D. Cells with co-overexpression of 3βHSD1-GST and HA-BMX or vehicle were treated with DHEA for 1 hour. Phospho-3βHSD1-Y344 was detected by immunoprecipitation and western blot. E. Cells overexpressing 3βHSD1-GST were transfected with siNT or one of two siRNA sequences against BMX; phospho-3βHSD1-Y344 was detected by GST pull-down and western blot. F. Cells with co-overexpression of 3βHSD1-GST and HA-BMX or HA-BMX-KD (kinase dead) were treated with DHEA for 1 hour. Phospho-3βHSD1-Y344 was detected by immunoprecipitation and western blot. G. 3βHSD1-GST or HA-BMX was purified from 293T cells; 3βHSD1-GST was dephosphorylated using phosphatase in vitro, followed by a kinase assay and western blot. H, I, J. 293T cells were transfected with 3βHSD1 or Y344F mutant with or without co-overexpressed HA-BMX. 3βHSD1 or 3βHSD1-Y344F mutant was immunopurified, and an NAD+ turnover assay was performed. K. GST-tagged and flag-tagged WT or Y344F-3βHSD1 were transfected into C4-2 cells. GST pull-down was performed, and flag-tagged 3βHSD1 was detected by western blot. DHEA was treated for 2 hours. L. GST-tagged and flag-tagged WT or Y344F-3βHSD1 were transfected into C4-2 cells. GST pull-down was performed, and flag-tagged 3βHSD1 was detected by western blot. Cells were treated with DHEA for 2 hours; zanubrutinib (10 μM) treatment was 24 hours. For all panels, unless otherwise noted, error bars represent the SEM; P values were calculated using un-paired two-tailed t tests. * P<0.05. ** P<0.01.

FIG. 4. BMX blockade and inhibition of 3βHSD1 phosphorylation inhibit expression of androgen-regulated genes and prostate cancer proliferation. A. C4-2 cells with stable shRNA-mediated knockdown of 3βHSD1 were stably infected with lentivirus expressing either 3βHSD1 (WT) or 3βHSD1-Y344F and subsequently treated with [³H]-DHEA for 5 hours, followed by steroid extraction from media and steroid separation and quantitation with HPLC. B. As in (A), but cells were deprived of serum overnight, followed by treatment with DHEA for the indicated days; cell proliferation was assessed with the WST-1 assay and growth for each cell line was normalized to WT control for each designated day. C. As in (A), but cells were deprived of serum overnight and treated with DHEA for 48 hours, followed by RNA extraction and qPCR. Expression is normalized to control and RPLP0 expression. Data are presented as the mean±SEM of biological quadruplicates and are representative of at least three independent experiments. * P<0.05. ** P<0.01. D. Cells stably expressing shNT or one of two shRNA sequences against BMX were deprived of serum overnight, followed by treatment with DHEA and cell proliferation assessment as in (B). E. As in (C), except that RNA extraction and qPCR was done on shBMX or control cells treated with DHEA for 48 hours. F. LNCaP or C4-2 cells were deprived of serum overnight, treated with zanubrutinib or DHEA for the indicated times, and cell proliferation assessed as in (B). G. LNCaP or C4-2 cells were deprived of serum overnight and treated with zanubrutinib or DHEA for 48 hours, followed by RNA extraction and qPCR. Expression is normalized to control and RPLP0 expression. Error bars represent the SEM; P values were calculated using un-paired two-tailed t tests. * P<0.05. ** P<0.01.

FIG. 5. 3βHSD1-Y344F blocks CRPC growth in vivo. A. C4-2 cells with stable shRNA-mediated knockdown of 3βHSD1 were stably infected with a lentivirus expressing 3βHSD1 (WT) or 3βHSD1-Y344F. Mice were subcutaneously injected with 10 million cells, and castration plus DHEA pellet implantation were performed after tumors reached 200 mm3. Tumor growth is shown as fold change from the time of treatment initiation for each tumor. The numbers of mice in the WT-3βHSD1/castration and 344F-3βHSD1/castration groups were 13 and 12, respectively. B. Progression-free survival was assessed as time to 3-fold increase in tumor volume from treatment initiation, and the significance of the difference was calculated with a log-rank test. C. The testosterone concentration in xenograft tumors was detected by mass spectrometry. D. RNA was extracted from xenograft tumors, expression of AR responsive genes (PSA, FKBP5 and TMPRSS2) and HSD3B1 was determined by qPCR. Expression is normalized to control and RPLP0 expression. E. C4-2 cells with stable shRNA-mediated knockdown of 3βHSD1 were stably infected with a lentivirus expressing 3βHSD1 (WT) or 3βHSD1-Y344F, and xenograft experiments were initiated as in (A) except mice were not castrated. The numbers of mice in the WT-3βHSD1/eugondal or 344F-3βHSD1/eugonadal groups were 12 and 11, respectively. F. Progression-free survival was assessed as time to 3-fold increase in tumor volume from the time tumors reached 200 mm3. G. Testosterone concentration in xenograft tumors was detected by mass spectrometry. H. RNA was extracted from xenograft tumors and gene expression was determined by qPCR, as in (D). For all panels, unless otherwise noted, error bars represent the SEM; P values were calculated using un-paired two-tailed t tests.

FIG. 6. BMX pharmacologic blockade impedes 3βHSD1-driven CRPC growth in vivo. A. Six million C4-2 cells were injected subcutaneously in mice, and castration. DHEA pellet implantation and treatment with vehicle or zanubrutinib at a dose of 15 mg/kg by oral gavage twice daily was performed after tumors reached 150 mm³. Tumor growth was assessed as fold change from time of treatment initiation. The numbers of mice in the castration/vehicle and castration/zanubrutinib groups were 13 and 12, respectively. B. Progression-free survival was assessed as time to 3-fold increase in tumor volume from treatment initiation, and the statistical difference was calculated with a log-rank test. C. Tumor testosterone in xenograft tumors was detected by mass spectrometry. D. Expression of AR-regulated genes was assessed by qPCR and expression is normalized to control and RPLP0. E. RNA-seq and gene set enrichment analysis was performed, showing AR inhibition as the top upstream regulator predicted as inhibited. F. Ten million VCaP cells were injected subcutaneously. Castration. DHEA pellet implantation, and vehicle or zanubrutinib at a dose of 15 mg/kg by oral gavage twice daily were performed after tumors reached 200 mm³, and fold-change in tumor volume from the time of treatment initiation was assessed. The numbers of mice in the castration/vehicle and castration/zanubrutinib groups were 12 and 11, respectively. G. Progression-free survival was assessed in (B). H. Xenograft testosterone detection by mass spectrometry. I. AR-regulated genes were assessed as in (D). For all panels, unless otherwise noted, error bars represent the SEM; P values were calculated using un-paired two-tailed t tests.

FIG. 7. Targeting BMX inhibits phosphorylation and enzymatic activity of 3βHSD1 in prostate tissue of prostate cancer patients. A. Fresh prostate tissues from 3 representative examples of prostate cancer patients that exhibited DHEA metabolism (MT, homozygous HSD3B1(1245C); HZ, heterozygous). Tissues were obtained, and aliquoted in two equal portions. One was treated with zanubrutinib, and the other with DMSO. Both portions were maintained in 3 ml DMEM containing 10% fetal bovine serum, incubated for 12 hours and then [³H]-DHEA was added to each portion. Cell culture medium was collected at the indicated times, and HPLC was performed. B. DHEA metabolism was analyzed on day 7 and day 15. C. Protein was extracted from about 20 mg patient tissue, followed by 3βHSD1 immunoprecipitation and western blot. D. The remaining tissue was used for western blot: tissue cores were minced and aliquoted in 2 equal parts and treated as in (A). After 12 hours of culture. DHEA (10 nM) was added to each portion. Seven days later, protein was collected, and immunoprecipitation and western blot were performed. E. Proposed model for 3βHSD1 phosphorylation. BMX phosphorylates 3βHSD1 Y344 upon substrate activation Y344 phosphorylation enhances 3βHSD1 activity by increasing its dimerization, which subsequently promotes androgen production and prostate cancer proliferation. When BMX is inhibited. 3βHSD1-phosphorylation-stimulated dimerization is lost, reducing cellular enzyme activity, potent androgen production, and prostate cancer proliferation. For all panels, error bars represent the SEM; P values were calculated using paired two-tailed t tests.

FIG. 8. 3βHSD1 phosphorylation. A. C4-2 cells overexpressing HA-3βHSD1 were treated with steroids for 1 hour. Pan-phospho-tyrosine (pTyr) was detected by immunoprecipitation and western blot. B. LNCaP cells overexpressing HA-3βHSD1 were treated with DHEA for the indicated times. Pan-phospho-tyrosine (pTyr) was detected by immunoprecipitation and western blot. C. 3βHSD1-GST was transfected, and cells were treated with DHEA for 1 hour. GST pull-down complexes were immunoblotted, and the indicated phosphopeptides on human 3βHSD1 were identified by LC-MS/MS. A triply charged peptide with a mass of 983.75 Da was identified in a targeted analysis of GST-HSD3B1. The CID spectra for this peptide are dominated by doubly charged C-terminal y ions. The mass difference between y₁₉ and y₂₀ is consistent with modification at Y255. D. Chromatograms for the unmodified. Y255, and pY255 peptides from GST-HSD3B1 are shown. Chromatograms for the unmodified. Y344, and pY344 peptides from GST-HSD3B1 are shown. E. HA-3βHSD1 enzyme activity was assessed by determining DHEA metabolism with HPLC. Cells were transfected with HA-3βHSD1 mutants and subsequently treated with [³H]-DHEA for 6 hours, followed by steroid extraction from media and steroid separation and quantitation with HPLC. ** P<0.01 F. Validation of phospho-3βHSD1-Y344 antibody.

FIG. 9. Tyrosine kinase inhibitors and regulation of cellular 3βHSD1 activity. A. BMX inhibition suppresses DHEA metabolism. Kinase screening was performed by detecting DHEA metabolism using a kinase inhibitor library. LNCaP cells were treated with about 88 kinase inhibitors (10 μM) for 1 hour and subsequently treated with [³H]-DHEA for 5 hours. Medium was then collected, and steroids were extracted and separated and quantified with HPLC. Color scale indicates % DHEA remaining. B. LNCaP or C4-2 cells were treated with BMX inhibitors for 1 hour and subsequently treated with [³H]-DHEA for 5 hours. Medium was then collected and steroids were extracted from medium and separated and quantified with HPLC. C, D. As in (B), but experiments were done in both LNCaP and VCaP cell lines using various concentrations of zanubrutinib for the indicated times to determine effects on DHEA metabolism. E. JEG3 cells were starved with RPMI-1640 medium containing 10% charcoal-stripped fetal bovine serum; 24 hours later, 10 μM zanubrutinib was added, followed by DHEA 16 hours later. After DHEA addition, cells were treated for the indicated times, medium was collected, and mass spectrometry was performed to detect the steroid level. * P<0.05. ** P<0.01, and *** P<0.001.

FIG. 10. Effects of 3βHSD1-Y344F mutation on protein levels. A. C4-2 cells with stable shRNA-mediated knockdown of 3βHSD1 were stably infected with a lentivirus expressing 3βHSD1 (WT) or 3βHSD1-Y344F grown in castrated mice after tumors reached 200 mm³. Proteins were extracted from tumor tissue, and western blot was performed. B. C4-2 cells were treated with 2, 5, or 10 μM zanubrutinib for 48 hours. Proteins were extracted, and western blot was performed. C. Flag-tagged WT or Y344F-3βHSD1 was transfected into C4-2 cells; then cells were treated with cyclohexamide (CHX) for the indicated times, followed by protein extraction, western blot, and protein quantitation.

FIG. 11. Effects of BMX inhibition on cell viability. A. LNCaP cells were deprived of serum overnight, followed by treatment with ibrutinib or DHEA for the indicated times, and the cell number was determined using Trypan blue cell counting. B. C4-2 cells were deprived of serum overnight, followed by treatment with ibrutinib or DHEA for the indicated times, and the cell number was determined with Trypan blue.

FIG. 12. Pharmacologic BMX inhibition and effects on xenografts in eugonadal mice. A. Six million C4-2 cells were injected subcutaneously in mice. After tumors reached 150 mm³, zanubrutinib or vehicle treatment was started. Tumor growth was analyzed. The numbers of mice in the eugonadal/vehicle, eugonadal/zanubrutinib groups were 11, 12 respectively. B, progression-free survival was assessed as time to 3-fold increase in tumor volume from treatment initiation and comparison between the groups was calculated with a log-rank test. C. Absolute concentration of testosterone in xenograft tumors were detected by mass spectrometry. D. RNA was extracted from xenograft tumors and AR target gene expression was determined by qPCR. Expression is normalized to control and RPLP0 expression. E. RNA-seq was performed with 4 tumors from each group and Gene Set Enrichment Analysis was done for the AR signature gene set. F. Ten million VCaP cells were injected subcutaneously in mice and treatment with vehicle or zanubrutinib was initiated when tumor volume reached 200 mm³. There were 11 mice in each cohort for the eugonadal/vehicle, eugonadal/zanubrutinib groups. G. Progression-free survival was assessed as time to 3-fold increase in tumor volume from treatment initiation and the significance of the difference between groups was calculated with a log-rank test. H. Testosterone in xenograft tumors was detected by mass spectrometry. I. Expression of AR target gene expression was performed as in (D).

FIG. 13. Effects of zanubrutinib on 3βHSD1 activity in human prostate tissue. Fresh prostate tissue cores (40-60 mg) from 4 patients were obtained, minced and aliquoted in two portions. One was treated with zanubrutinib, and the other was treated with DMSO. Both tissues were maintained in 3 ml DMEM containing 10% fetal bovine serum and incubated in a 5% CO2 humidified incubator. After 12 hours of culture, [³H]-DHEA was added to each portion. Cell culture medium was collected, and HPLC was performed.

### DEFINITIONS

For convenience, certain terms employed in the specification, examples, and appended claims are collected here. Unless defined otherwise, all technical and scientific terms used herein have the same meaning as commonly understood by one of ordinary skill in the art to which this invention belongs.

The phrases “parenteral administration” and “administered paternterally” as used herein means modes of administration other than enteral and topical administration, usually by injection, and includes, without limitation, intravenous, intramuscular, intraaterial, intrathecal, intraventricular, intracapsular, intraorbital, intracarida, intradermal, intraperitoneal, transtracheal, subcutaneous, subcuticular, intraarticular, subcapsular, subarachnoid, intraspinal and transternal injection and infusion. The BMX inhibitor compositions of the present invention may be administered by parenteral administration.

The phrases “systemic administration,” administered systemically,” “peripheral administration,” and “administered peripherally” as used herein mean the administration of a compound, drug or other material other than directly into the central nervous system, such that it enters the animal's system and, thus, is subject to metabolism and other like processes, for example, subcutaneous administration. The BMX inhibitor compositions of the present invention may be administered by systemic administration.

### DETAILED DESCRIPTION

The present invention provides methods, kits, and compositions for treating sex steroid dependent cancer using BMX inhibitors. In certain embodiments, a sample from a subject having, or suspected of having, sex steroid dependent cancer, is further assayed to determine: i) if the subject is heterozygous or homozygous for the HSD3B1(1245C) allele that encodes for the 3βHSD1(367T) protein. In some embodiments, the BMX inhibitor comprises a monoclonal antibody or BMX binding portion thereof (e.g., zanubrutinib, acalabrutinib, abivertinib or ibrutinib).

The BMX inhibitors herein may be formulated in a pharmaceutical composition. The pharmaceutical composition can include a pharmaceutically acceptable carrier and a non-toxic therapeutically effective amount of the compositions of the present invention. The phrases “pharmaceutically or pharmacologically acceptable” refer to molecular entities and compositions that do not produce an adverse, allergic, or other untoward reaction when administered to an animal, or a human, as appropriate. Veterinary uses are equally included within the invention and “pharmaceutically acceptable” formulations include formulations for both clinical and/or veterinary use. As used herein, “pharmaceutically acceptable carrier” includes any and all solvents, dispersion media, coatings, antibacterial, and antifungal agents, isotonic and absorption delaying agents and the like. The use of such media and agents for pharmaceutical active substances is well known in the art. Except insofar as any conventional media or agent is incompatible with the active ingredient, its use in the therapeutic compositions is contemplated. For human administration, preparations should meet sterility, pyrogenicity, general safety and purity standards as required by FDA Office of Biologics standards. Supplementary active ingredients can also be incorporated into the compositions.

Examples of carriers include solvents and dispersion media containing, for example, water, ethanol, polyol (for example, glycerol, propylene glycol, and liquid polyethylene glycol, and the like), mixtures thereof, and vegetable oils. In many cases, it will be preferable to include isotonic agents, for example, by the use of a coating, such as lecithin, by the maintenance of the required particle size in the case of dispersion and/or by the use of surfactants.

The present invention contemplates the administration of the described pharmaceutical compositions by various routes. Pharmaceutical compositions comprising the compositions of the present invention may be administered by any route that ensures bioavailability in the circulation. These routes can include, but are by no means limited to parenteral administration, systemic administration, oral administration, nasal administration, rectal administration, intraperitoneal injection, intravascular injection, subcutaneous injection, transcutaneous administration, inhalation administration, and intramuscular injection.

Injectable preparations include sterile suspensions, solutions or emulsions of the active ingredient in aqueous or oily vehicles. The compositions may also contain formulating agents, such as suspending, stabilizing and/or dispersing agent. The formulations for injection may be presented in unit dosage form, e.g. in ampoules or in multidose containers, and may contain added preservatives.

Alternatively, the injectable formulation may be provided in powder form for reconstitution with a vehicle, including but not limited to sterile pyrogen free water, buffer, dextrose solution, etc., before use. To this end the compositions of the present invention may be lyophilized, or the co-lyophilized peptide-lipid complex may be prepared. The stored preparations can be supplied in unit dosage forms and reconstituted prior to use in vivo.

For prolonged delivery, the active ingredient can be formulated as a depot preparation, for administration by implantation; e.g., subcutaneous, intradermal, or intramuscular injection. Thus, for example, the active ingredient may be formulated with polymeric or hydrophobic materials (e.g., as an emulsion in an acceptable oil) or ion exchange resins, or as sparingly soluble derivatives. Alternatively, transdermal delivery systems manufactured as an adhesive disc or patch which slowly releases the active ingredient for percutaneous absorption may be used. To this end, permeation enhancers may be used to facilitate transdermal penetration of the active ingredient.

For oral administration, the pharmaceutical compositions may take the form of, for example, tablets or capsules prepared by conventional means with pharmaceutically acceptable excipients, such as binding agents (e.g., pregelatinised maize starch, polyvinylpryrrolidone or hydroxypropyl methylcellulose); fillers (e.g., lactose, microcrystalline cellulose or calcium hydrogen phosphate); lubricants (e.g., magnesium stearate, talc, or silica); disintegrants (e.g., potato starch or sodium starch glycolate); or wetting agents (e.g., sodium lauryl sulfate). The tablets may be coated by methods well known in the art. Liquid preparations for oral administration may take the form of, for example, solutions, syrups or suspensions, or they may be presented as a dry product for constitution with water or other suitable vehicle before use. Such liquid preparations may be prepared by conventional means with pharmaceutically acceptable additives, such as suspending agents (e.g., sorbitol syrup, cellulose derivatives or hydrogenated edible fats); emulsifying agents (e.g., lecithin or acacia); non-aqueous vehicles (e.g., almond oil, oily ester, ethyl alcohol or fractioned vegetable oils); and preservatives (e.g., methyl or propyl-p-hydroxy benzoates or ascorbic acid). The preparations may also contain buffer salts, flavoring, coloring, and sweetening agents as appropriate. Preparations for oral administration may be suitably formulated to give controlled release of the active compound.

For buccal administration, the compositions may take the form of tablets or lozenges formulated in conventional manner. For rectal and vaginal routes of administration, the active ingredient may be formulated as solutions (for retention enemas) suppositories or ointments.

For administration by inhalation, the active ingredient can be conveniently delivered in the form of an aerosol spray presentation from pressurized packs or a nebulizer, with the use of a propellant, e.g., dichlorodifluoromethane, trichlorofluoromethane, dichlorotetrafluoroethane, carbon dioxide or other gas. In the case of a pressurized aerosol, the dosage unit may be determined by providing a valve to deliver a metered amount. Capsules and cartridges of e.g. gelatin for use in an inhaler or insufflators may be formulated containing a powder mix of the compound and a suitable powder base such as lactose or starch.

The compositions may, if desired, be presented in a pack or dispenser device, which may contain one or more unit of dosage forms containing the active ingredient. The pack may for example comprise metal or plastic foil, such as a blister pack. The pack or dispenser device may be accompanied by instructions for administration.

“Unit dosage” formulations are those containing a dose or sub-dose of the administered ingredient adapted for a particular timed delivery. For example, exemplary “unit dosage” formulations are those containing a daily dose or unit or daily sub-dose or a weekly dose or unit or weekly sub-dose and the like.

In certain embodiments, the BMX inhibitor comprises one or more of the shRNA sequences shown in Table 1 below.

TABLE 1

| | | | Sigma Clone |
|---|---|---|---|
| | | | ID = TRC |
| | | | number |
| Gene | | | (from Sigma's |
| name | shRNA Sequence | Species | website) |
| BMX | GATCACAATCT | human | TRCN0000196600 |
| | GAACAGTTAC | | |
| | (SEQ ID NO: 1) | | |
| BMX | GAGTGCTGATA | human | TRCN0000006359 |
| | AGAATGAATA | | |
| | (SEQ ID NO: 2) | | |
| BMX | GCAATATGACA | human | TRCN0000006362 |
| | GCAACTCAAA | | |
| | (SEQ ID NO: 3) | | |
| BMX | CGTGCATACAA | human | TRCN0000006360 |
| | ATGCTGAGAA | | |
| | (SEQ ID NO: 4) | | |

In some embodiments, nucleic acid sequencing methods are utilized for detection of position 1245 in the HSD3B1 gene. In some embodiments, the sequencing technology employed is Second Generation (a.k.a. Next Generation or Next-Gen), Third Generation (a.k.a. Next-Next-Gen), or Fourth Generation (a.k.a. N3-Gen) sequencing technology including, but not limited to, pyrosequencing, sequencing-by-ligation, single molecule sequencing, sequence-by-synthesis (SBS), semiconductor sequencing, massive parallel clonal, massive parallel single molecule SBS, massive parallel single molecule real-time, massive parallel single molecule real-time nanopore technology, etc. Morozova and Marra provide a review of some such technologies in Genomics, 92: 255 (2008), herein incorporated by reference in its entirety. Those of ordinary skill in the art will recognize that because RNA is less stable in the cell and more prone to nuclease attack experimentally RNA is usually reverse transcribed to cDNA before sequencing.

In some embodiments, hybridization methods are utilized for detecting position 1245C. Illustrative non-limiting examples of nucleic acid hybridization techniques include, but are not limited to, in situ hybridization (ISH), microarray, and Southern or Northern blot.

In some embodiments, nucleic acid sequences are amplified (e.g., after conversion to DNA) prior to or simultaneous with detection. Illustrative non-limiting examples of nucleic acid amplification techniques include, but are not limited to, polymerase chain reaction (PCR), reverse transcription polymerase chain reaction (RT-PCR), transcription-mediated amplification (TMA), ligase chain reaction (LCR), strand displacement amplification (SDA), and nucleic acid sequence based amplification (NASBA). Those of ordinary skill in the art will recognize that certain amplification techniques (e.g., PCR) require that RNA be reversed transcribed to DNA prior to amplification (e.g., RT-PCR), whereas other amplification techniques directly amplify RNA (e.g., TMA and NASBA).

In some embodiments, quantitative evaluation of the amplification process in real-time is performed. Evaluation of an amplification process in “real-time” involves determining the amount of amplicon in the reaction mixture either continuously or periodically during the amplification reaction, and using the determined values to calculate the amount of target sequence initially present in the sample. A variety of methods for determining the amount of initial target sequence present in a sample based on real-time amplification are well known in the art. These include methods disclosed in U.S. Pat. Nos. 6,303,305 and 6,541,205, each of which is herein incorporated by reference in its entirety. Another method for determining the quantity of target sequence initially present in a sample, but which is not based on a real-time amplification, is disclosed in U.S. Pat. No. 5,710,029, herein incorporated by reference in its entirety.

Amplification products may be detected in real-time through the use of various self-hybridizing probes, most of which have a stem-loop structure. Such self-hybridizing probes are labeled so that they emit differently detectable signals, depending on whether the probes are in a self-hybridized state or an altered state through hybridization to a target sequence. By way of non-limiting example. “molecular torches” are a type of self-hybridizing probe that includes distinct regions of self-complementarity (referred to as “the target binding domain” and “the target closing domain”) which are connected by a joining region (e.g., non-nucleotide linker) and which hybridize to each other under predetermined hybridization assay conditions. In certain embodiments, molecular torches contain single-stranded base regions in the target binding domain that are from 1 to about 20 bases in length and are accessible for hybridization to a target sequence present in an amplification reaction under strand displacement conditions. Under strand displacement conditions, hybridization of the two complementary regions, which may be fully or partially complementary, of the molecular torch is favored, except in the presence of the target sequence, which will bind to the single-stranded region present in the target binding domain and displace all or a portion of the target closing domain. The target binding domain and the target closing domain of a molecular torch include a detectable label or a pair of interacting labels (e.g., luminescent/quencher) positioned so that a different signal is produced when the molecular torch is self-hybridized than when the molecular torch is hybridized to the target sequence, thereby permitting detection of probe:target duplexes in a test sample in the presence of unhybridized molecular torches. Molecular torches and a variety of types of interacting label pairs, including fluorescence resonance energy transfer (FRET) labels, are disclosed in, for example U.S. Pat. Nos. 6,534,274 and 5,776,782, each of which is herein incorporated by reference in its entirety.

Another example of a detection probe having self-complementarity is a “molecular beacon.” Molecular beacons include nucleic acid molecules having a target complementary sequence, an affinity pair (or nucleic acid arms) holding the probe in a closed conformation in the absence of a target sequence present in an amplification reaction, and a label pair that interacts when the probe is in a closed conformation. Hybridization of the target sequence and the target complementary sequence separates the members of the affinity pair, thereby shifting the probe to an open conformation. The shift to the open conformation is detectable due to reduced interaction of the label pair, which may be, for example, a fluorophore and a quencher (e.g., DABCYL and EDANS). Molecular beacons are disclosed, for example, in U.S. Pat. Nos. 5,925,517 and 6,150,097, herein incorporated by reference in its entirety.

In certain embodiments, position 367T or 367N are detected in a 3βHSDC protein in a sample from a subject. Detection techniques employed include, but not limited to: protein sequencing and immunoassays. Illustrative non-limiting examples of protein sequencing techniques include, but are not limited to, mass spectrometry and Edman degradation.

Mass spectrometry can, in principle, sequence any size protein but becomes computationally more difficult as size increases. A protein is digested by an endoprotease, and the resulting solution is passed through a high pressure liquid chromatography column. At the end of this column, the solution is sprayed out of a narrow nozzle charged to a high positive potential into the mass spectrometer. The charge on the droplets causes them to fragment until only single ions remain. The peptides are then fragmented and the mass-charge ratios of the fragments measured. The mass spectrum is analyzed by computer and often compared against a database of previously sequenced proteins in order to determine the sequences of the fragments. The process is then repeated with a different digestion enzyme, and the overlaps in sequences are used to construct a sequence for the protein.

In the Edman degradation reaction, the peptide to be sequenced is adsorbed onto a solid surface (e.g., a glass fiber coated with polybrene). The Edman reagent, phenylisothiocyanate (PTC), is added to the adsorbed peptide, together with a mildly basic buffer solution of 12% trimethylamine, and reacts with the amine group of the N-terminal amino acid. The terminal amino acid derivative can then be selectively detached by the addition of anhydrous acid. The derivative isomerizes to give a substituted phenylthiohydantoin, which can be washed off and identified by chromatography, and the cycle can be repeated. The efficiency of each step is about 98%, which allows about 50 amino acids to be reliably determined.

Illustrative non-limiting examples of immunoassays include, but are not limited to: immunoprecipitation; Western blot; ELISA; immunohistochemistry; immunocytochemistry; flow cytometry; and, immuno-PCR. Polyclonal or monoclonal antibodies detectably labeled using various techniques known to those of ordinary skill in the art (e.g., colorimetric, fluorescent, chemiluminescent or radioactive) are suitable for use in the immunoassays.

Immunoprecipitation is the technique of precipitating an antigen out of solution using an antibody specific to that antigen. The process can be used to identify protein complexes present in cell extracts by targeting a protein believed to be in the complex. The complexes are brought out of solution by insoluble antibody-binding proteins isolated initially from bacteria, such as Protein A and Protein G. The antibodies can also be coupled to sepharose beads that can easily be isolated out of solution. After washing, the precipitate can be analyzed using mass spectrometry, Western blotting, or any number of other methods for identifying constituents in the complex.

A Western blot, or immunoblot, is a method to detect protein in a given sample of tissue homogenate or extract. It uses gel electrophoresis to separate denatured proteins by mass. The proteins are then transferred out of the gel and onto a membrane, typically polyvinyldiflroride or nitrocellulose, where they are probed using antibodies specific to the protein of interest. As a result, researchers can examine the amount of protein in a given sample and compare levels between several groups.

An ELISA, short for Enzyme-Linked ImmunoSorbent Assay, is a biochemical technique to detect the presence of an antibody or an antigen in a sample. It utilizes a minimum of two antibodies, one of which is specific to the antigen and the other of which is coupled to an enzyme. The second antibody will cause a chromogenic or fluorogenic substrate to produce a signal. Variations of ELISA include sandwich ELISA, competitive ELISA, and ELISPOT. Because the ELISA can be performed to evaluate either the presence of antigen or the presence of antibody in a sample, it is a useful tool both for determining serum antibody concentrations and also for detecting the presence of antigen.

### EXAMPLES

### Example 1

### BMX Controls 3βHSD1 and Extragonadal Sex Steroid Biosynthesis in Cancer

In this Example, we identified multiple phosphorylation sites on 3βHSD1 and found that phosphorylation of a single tyrosine (Y) is required for 3βHSD1 activation and that BMX is the necessary kinase. BMX blockade inhibits metabolism of (→⁵, 3β-OH) DHEA to (Δ⁴, 3-keto)→⁴-androstenedione (AD), which is the major substrate for steroid-5α-reductase (SRD5A) and aromatase, downstream enzymes required for potent androgen and estrogen synthesis, respectively. BMX therefore regulates the generation of the Δ⁴, 3-keto-steroid structure, which is necessary for all potent sex steroids. Further, we show that targeting BMX is a potential treatment strategy for CRPC.

### Methods and Materials

### Antibodies, Chemicals, and Reagents

Antibodies: Mouse monoclonal antibodies against 3βHSD1(1:2000, ab55268) and rabbit polyclonal antibodies against phospho-BMX (1:2000, #ab59409) were purchased from Abcam (Waltham, MA, USA). Mouse monoclonal antibodies against phospho-tyrosine (pTyr) (1:2000, 05-1050) were purchased from Sigma (Burlington, MA, USA). Rabbit monoclonal antibodies against phospho-3βHSD1 Y344 (1:2000) were ordered from Abcite (Shanghai, China). Mouse monoclonal antibodies against GST (1:5000, AE001) were purchased from Abclonal (Shanghai, China). Mouse monoclonal antibodies against Flag (1:5000, F3165) and anti-Flag M2 affinity gel (A2220) were purchased from Sigma (St. Louis, MO, USA). Rabbit monoclonal antibodies against HA (1:3000, 3724S), β-actin (1:3000,3700S), and rabbit polyclonal antibodies against BMX (1:3000, 24773) and GAPDH (1:5000, 14C10) were obtained from Cell Signaling Technology (Danvers, MA, USA).
Chemicals: Zanubrutinib (BGB-3111), ibrutinib (9680), and acalabrutinib (ACP-196) were purchased from Selleckchem (Thousand Oaks, CA, USA). The kinase inhibitor library was obtained from the Lerner Research Institute Molecular Screening Core. [³H]-labeled DHEA (100 nM, 300,000-600,000 cpm) was purchased from PerkinElmer (Waltham, MA), and steroids were purchased from Steraloids (Newport, RI, USA).
Reagents: Puromycin (A1113803) and hygromycin (10687010) were bought from ThermoFisher Scientific (Waltham, MA, USA). DNA transfection reagent FuGENE HD (E2311) was purchased from Promega (USA). GelCode Blue Stain Reagent (24590) was obtained from Pierce (Rockford, IL, USA).

### Cell Lines and Constructs

LNCaP and C4-2 cells were purchased from the American Type Culture Collection (ATCC) (Manassas, VA) and cultured in RPMI 1640 medium with 10% fetal bovine serum. VCaP, JEG3 and 293T cells were purchased from ATCC and cultured in DMEM containing 10% fetal bovine serum. LAPC4 cells were a gift from Charles Sawyers (Memorial Sloan Kettering Cancer, New York, NY) and were maintained in Iscove's modified Dulbecco's medium with 10% fetal bovine serum (Gemini) and 1% penicillin-streptomycin (Gibco). Constructs of shRNAs targeting BMX (5′-GCAATATGACAGCAACTCAAA-3′ (SEQ ID NO:5): 5′-GATCACAATCTGAACAGTTAC-3′ (SEQ ID NO:6)) and targeting HSD3B1 (5′-GAAGGTTTCTGTCCTAATCAT-3′ (SEQ ID NO:7) were purchased from Sigma. These constructs were used to generate the BMX knockdown LNCaP stable cell lines or HSD3B1 knockdown C4-2 stable cell lines by using a lentiviral system. 293T cells (ATCC) were cotransfected for 48 hours with 10 μg each of the constructed plasmid, pMD2.G, and psPAX2 vector to package the virus. The virus was then concentrated by using PEG-it Virus Precipitation Solution (System Biosciences) according to the provided protocol. Next, LNCaP or C4-2 cells were infected with the concentrated virus for 24 hours with addition of polybrene (10 μg/ml), followed by selection with puromycin (2 μg/ml) for ˜2 weeks.

pCMV5-HA-HSD3B1 was kindly provided by J. Ian Mason (Lorence et al., 1990), sequenced, and confirmed as encoding for 3βHSD1(367T). PCR-amplified 3βHSD1(367T) was cloned into pCMV-Flag, pCDH-GST. The plasmid encoding mutated 3βHSD1(Y344F, Y255F, Y340F) was derived by using the Quick Change Site Directed Mutagenesis kit (Agilent Technologies, Santa Clara, CA). For C4-2 cells stably expressing 3βHSD1 (WT) or 3βHSD1-Y344F, the wild type or mutant was PCR-amplified and sub-cloned into the pLVX-Flag-Puro vector. Lentiviral particles were packaged in 293T cells by co-transfecting 10 μg each of pLVX-Flag-Puro vector, pMD2.G, and psPAX2 vector. Next, C4-2 cells with stable shRNA-mediated knockdown of 3βHSD1 were stably infected with the concentrated virus for 24 hours with addition of polybrene (10 μg/ml), followed by selection with hygromycin (2 μg/ml) for ˜2 weeks.

A guide RNA sequence for targeting HSD3B1 5′-CGTTTATACTAGCAGAAAGGC-3′ (SEQ ID NO:8) was designed and cloned, and virus was produced using the LentiCRISPRv2 protocol (Sanjana et al., 2014).

### Steroid Metabolism

Cells (300,000-400,000 cells per well) were seeded and maintained in 12-well plates that were coated with poly-L-ornithine (Sigma-Aldrich) for 12 hours and then treated with [³H]-DHEA (100 nM, 300,000-600,000 cpm; PerkinElmer). Cells were incubated at 37° ° C. and aliquots of medium (0.3 ml) were collected at the indicated time points. Collected media was incubated with 300 U β-glucuronidase (Helix pomatia; Novoprotein) at 37ºC for at least 2 hours, extracted with 600 μl 1:1 ethyl acetate:isooctane, and concentrated under a nitrogen stream.

For high-performance liquid chromatography (HPLC) analysis, the concentrated samples were dissolved in 50% methanol and injected on a Breeze 1525 system equipped with a model 717 plus autoinjector (Waters Corp.). Steroid metabolites were separated on a Luna 150×4.6 mm, 3 μm C18 reverse-phase column (Phenomenex) using methanol/water gradients at 30° C. The column effluent was analyzed using a β-RAM model 3 in-line radioactivity detector (IN/US Systems Inc.) with Liquiscint scintillation mixture (National Diagnostics). All metabolism studies were performed in triplicate and repeated in independent experiments.

### Gene Expression

Total RNA was extracted with GenElute Mammalian Total RNA miniprep kit (Sigma-Aldrich), and 1 μg RNA was reverse-transcribed to complementary DNA (cDNA) with the iScript cDNA Synthesis Kit (Bio-Rad). An ABI 7500 Real-Time PCR instrument (Applied Biosystems) was used to perform quantitative PCR (qPCR) analysis, using iTaq Fast SYBR Green Supermix with ROX (Bio-Rad) in 96-well plates at a final reaction volume of 10 μl. The qPCR analysis was carried out in triplicate with the following primer sets:

HSD3B1:
(SEQ ID NO: 9)
5′-CCATGTGGTTTGCTGTTACCAA-3′
(forward)
and
5′-TCAAAACGACCCTCAAGTTAAAAGA-3′
(SEQ ID NO: 10)
(reverse);
PSA:
5′-GCATGGGATGGGGATGAAGTAAG-3′
(SEQ ID NO: 11)
(forward)
and
5′-CATCAAATCTGAGGGTTGTCTGGA-3′
(SEQ ID NO: 12)
(reverse);
FKBP5:
5′-CCCCCT-GGTGAACCATAATACA-3′
(SEQ ID NO: 13)
(forward)
and
5′-AAAAGGCCACCT AGCTTTTTGC-3′
(SEQ ID NO: 14)
(reverse);
TMPRS9:
5′- CCATTTGCAGGATCTGTCTG-3′
(SEQ ID NO: 15)
(forward)
and
5′-GGATGTGTCTTGGGGAGCAA-3′
(SEQ ID NO: 16)
(reverse);

RPLP0 (large ribosomal protein P0, a housekeeping gene):

(SEQ ID NO: 17)
5′-CGAGGGCAC
CTGGAAAAC-3′
(forward)
and
(SEQ ID NO: 18)
5′-CACATTCCCCCG-
GATATGA-3′
(reverse)

For steroid-treated cells, each mRNA transcript was quantitated by normalizing the sample values to RPLP0 and to vehicle-treated cells. All gene expression studies were repeated in at least three independent experiments.

### Immunoblots and Immunoprecipitation

For immunoblots, total cellular protein was extracted with ice-cold radioimmunoprecipitation assay (RIPA) lysis buffer (Sigma-Aldrich) containing protease inhibitors (Roche) and phosphatase inhibitors (Sigma-Aldrich). Protein concentration was determined using a BCA protein assay (Pierce Protein Research Products, Thermo Fisher Scientific). Protein, 30 μg to 50 μg, was separated by 10% SDS-polyacrylamide gel electrophoresis and then transferred to a nitrocellulose membrane (Millipore). After incubating overnight at 4° C. with the anti-pTyr antibody, anti-BMX antibody, anti-p-BMX antibody, anti-3βHSD1 antibody, anti-GST antibody, or anti-flag antibody as appropriate, the appropriate secondary antibody was added and incubated for 1 hour at room temperature. A chemiluminescent detection system (Thermo Fisher Scientific) was used to detect the bands with peroxidase activity. An anti-GAPDH antibody (1:5000; Sigma-Aldrich) was used as a control for sample loading.

For immunoprecipitation of HA- or GST-tagged 3βHSD1, cell lysates (2 mg) were incubated with 30 μl anti-HA or anti-GST affinity gel overnight at 4° C. Beads were washed with lysis buffer four times and samples were then used for immunoblotting with phospho-Tyr or phospho-3βHSD1-Y344. Protein lysates, 50 μg each, were loaded on an SDS-polyacrylamide gel. For co-immunoprecipitation of 3βHSD1 and kinase, 293T cells (at 60% confluence) were transfected with 5 μg GST-tagged 3βHSD1 and HA-tagged kinase for 36 hours. Immunoprecipitation was performed as described above.

### Mass Spectrometry Analysis of 3βHSD1 Phosphorylation

Using an anti-GST affinity gel, GST-tagged 3βHSD1 was immunoprecipitated from C4-2 cells treated with DHEA (10 nM) for 1 hour. The precipitated complexes were boiled at 95ºC for 10 minutes. GST-tagged 3βHSD1 was separated from the complexes by SDS-PAGE and then trypsinized. The GST-tagged 3βHSD1 band was excised from the gel as closely as possible and washed and de-stained in 50% ethanol, 5% acetic acid. The gel pieces were then dehydrated in acetonitrile, dried in a Speed-vac, and digested by adding 5 μl trypsin (10 ng/μl) in 50 mM ammonium bicarbonate, followed by incubation overnight. The peptides were extracted into two portions of 30 μl each 50% acetonitrile, 5% formic acid. The combined extracts were evaporated to <10 μl in a Speed-vac and then re-suspended in 1% acetic acid to make up a final volume of ˜30 μl for LC-MS analysis.

The LC-MS system was a ThermoScientific Orbitrap Elite system. The HPLC column was a Dionex 15 cm×75 μm id Acclaim PepMap C18, 2 μm, 100 Å reversed-phase capillary chromatography column. Five microliters of the extract volume was injected, and the peptides, eluted from the column in an acetonitrile, 0.1% formic acid gradient at a flow rate of 0.25 μl/min, were introduced into the source of the mass spectrometer online. The micro-electrospray ion source was operated at 2.5 kV. The digest was analyzed in both a survey manner and a targeted manner. The survey experiments were performed using the data-dependent multitask capability of the instrument, acquiring full scan mass spectra to determine peptide molecular weights and product ion spectra to determine amino acid sequences in successive instrument scans. The LC-MS/MS data were searched with the program Sequest (bundled into Proteome Discoverer 2.3) against both the human UniProtKB database (downloaded on 2-28-2019, 20429 entries) and specifically against the sequence of GST-tagged 3βHSD1. The parameters used in this search include a peptide mass accuracy of 10 ppm, fragment ion mass accuracy of 0.6 Da, carbamidomethylated cysteines as a constant modification, and oxidized methionine and phosphorylation at S, T, and Y as a dynamic modification. The results were filtered to a peptide and protein level FDR rate of <1% using a target decoy strategy. All positively identified phosphopeptides were manually validated. The targeted experiments involve the analysis of specific GST-tagged 3βHSD1 peptides. The chromatograms for these peptides were plotted based on known fragmentation patterns, and the peak areas of these chromatograms were used to determine the extent of phosphorylation (Waitkus et al., 2014; Willard et al., 2003).

### In Vitro Kinase Assay

In brief, GST-3βHSD1 and HA-BMX were purified from 293T cells. 3βHSD1 was dephosphorylated by incubating with alkaline phosphatase at 37° C., then incubated with or without BMX in kinase buffer (60 mM HEPES pH 7.5, 5 mM MgCl2, 5 mM MnCl2, 3 M Na3VO4 and 1.25 mM DTT). ATP, 20 μM, was added to the kinase buffer to start the reaction. The reactions were performed in a total volume of 50 μl at 30° ° C. for 30 minutes and then terminated by adding SDS-PAGE loading buffer.

### Enzyme Kinetics

293T cells were transfected with Flag-3βHSD1 or Y344F mutant with or without co-overexpressed HA-BMX. Then 48 hours later, 3βHSD1 or 3βHSD1-Y344F mutant was purified using the FLAG M Purification Kit (Sigma-Aldrich, catalog no. CELLMM2) according to the protocol provided by the manufacturer. Briefly, cell pellets were washed with 10 volumes of phosphate-buffered saline and centrifuged. Cells were suspended in CelLytic M reagent and incubated for 20 minutes on ice. The cells were then centrifuged, and the supernatant was loaded onto the prepared column, which included anti-FLAG M2 affinity gel under gravity flow. The column was then washed with 10 column volumes of 1× wash buffer to remove unbound proteins, and then 3βHSD1 protein was eluted with 1 ml of 1× wash buffer containing 3×FLAG peptide (200 ng/ml). The Flag peptides were removed, and proteins were concentrated using an Amicon Ultra-0.5 centrifugal filter concentrator (Millipore) and quantitated by BCA protein assay (Pierce). 1 μg Flag-3βHSD1 or Y344F protein was subjected to SDS-PAGE. The protein purity was verified by GelCode Coomassie blue stain reagent (Pierce) following the instructions of the manufacturer and also verified by western blotting. For Coomassie blue staining, the gel was washed with deionized water for 15 minutes and then incubated with GelCode stain reagent for 1 hour followed by ultrapure water for 1 hour.

To detect the kinetics of 3βHSD1, an NAD+ turnover assay was performed. Preparations containing DHEA (1-20 μM), NAD+(0.1 mM) and 1 μg protein in 0.25 ml of 50 mM potassium phosphate (pH 7.4) were incubated for 1 hour before using the Promega NADH detection kit, After incubating an additional hour, luminescence was measured using a BioTek Synergy Neo Multi-Mod Plate Reader (BioTek, USA). The Km and Vmax were calculated by Michaelis-Menten analysis with nonlinear regression using GraphPad Prism software.

### Cell Proliferation Assay

Cells (˜10⁴/well) were plated in triplicate in 96-well plates coated with poly-dl-ornithine, incubated overnight, then starved with phenol red-free medium containing 5% charcoal: dextran-stripped fetal bovine serum for 48 hours and treated with 10 nM DHEA and combined with indicated drug treatments for 5 days, and assayed using the Cell Proliferation Reagent WST-1 (Sigma, USA). Absorbance was normalized to controls as indicated.

### Mouse Xenograft Studies

All mouse studies were performed under a protocol approved by the Institutional Animal Care and Use Committee of the Cleveland Clinic Lerner Research Institute. All NOD scid gamma (NSG) male mice (6 to 8 weeks old) were purchased from the Jackson Laboratory. Six to 10 million cells were injected subcutaneously in mice. After tumors reached 150-200 mm³, mice were surgically orchiectomized and implanted with 5 mg 90-day sustained-release DHEA pellets to mimic human adrenal DHEA production in men with CRPC.

To evaluate 3βHSD1 (WT) or 3βHSD1-Y344F C4-2 cell growth in vivo, 10 million 3βHSD1 (WT) or 3βHSD1-Y344F stable C4-2 cells (100 μl in 50% Matrigel and 50% growth media) were subcutaneously injected into mice. When tumors reached 200 mm³ (length× width× width×0.52), the mice were arbitrarily placed into two groups: eugonadal or castration plus DHEA treatment. Tumor volume was measured every other day, and progression-free survival was assessed as time to 3-fold increase in tumor volume from the time tumors reached 200 mm³. The numbers of mice in the 3βHSD1 (WT)/eugondal, 3βHSD1 (WT)/castration, 3βHSD1-Y344F/eugonadal or 3βHSD1-Y344F/castration groups were 12, 13, 11, and 12, respectively. The number of mice in each group was determined by those that survived surgical procedures and had reached the 200 mm³ tumor volume required to initiate treatment. Tumor diameters were measured by digital calipers three times per week. Tumors were fresh frozen upon mouse sacrifice.

To evaluate whether zanubrutinib suppresses tumor growth, 6 million C4-2 or 10 million VCaP cells (100 μl in 50% Matrigel and 50% growth media) were subcutaneously injected into mice. When tumors reached 150 (C4-2) or 200 (VCaP) mm³ (length× width×width×0.52), the mice were arbitrarily divided among four groups: eugonadal/vehicle [safflower seed oil (Sigma-Aldrich) with 10% dimethyl sulfoxide (DMSO)], eugonadal/zanubrutinib (15 mg/kg in safflower seed oil with 10% DMSO), castration/vehicle, and castration/zanubrutinib. The mice were given vehicle or zanubrutinib by oral gavage twice daily. Tumor volume and progression-free survival were determined as described above. The numbers of mice in the C4-2 eugonadal/vehicle, eugonadal/zanubrutinib, castration/vehicle and castration/zanubrutinib groups were 11, 12, 13 and 12, respectively. The numbers of mice in the VCaP eugonadal/vehicle, eugonadal/zanubrutinib, castration/vehicle and castration/zanubrutinib groups were 11, 11, 12, and 11, respectively. The number of mice in each group was determined by those that survived surgical procedures and had reached the 200 mm³ tumor volume required to initiate treatment. Tumor diameters were measured by digital calipers three times per week and fresh frozen upon mouse sacrifice.

### Mass Spectrometry Analysis

### Xenograft Tissues

Androgens in xenografts were assessed by liquid chromatography tandem mass spectrometry as reported previously with slight modifications (Li et al., 2021; Zhu et al., 2018). In brief, at least 30 mg tumor tissue was homogenized with 500 μL LC-MS-grade water (ThermoFisher Scientific) using a homogenizer. The mixture was then centrifuged at 15000×g for 10 minutes at 4° ° C. Supernatant was transferred to a glass tube, followed by the addition of 25 μL internal standard (d3-T). The steroids and the internal standard were extracted with 2 mL methyl tert butyl ether (Across) evaporated to dryness under nitrogen and then reconstituted with 200 μL 50% methanol.

### Estrogens and Androgens in Cell Culture

Steroid Extraction Freshly collected media samples were frozen and kept at −80° C., until the LC/MS/MS analysis. For the analysis, 250 μl media sample was spiked with 10 μl internal standards mix [5 ng/ml of E2-13C₃, 25 ng/ml, androstene-3, 17-dione-2,3,4-13C; and 5α-dihydrotestosterone-d3 (16,17,17-d3)] in a glass tube. The steroids were extracted using methyl-tert-butyl ether (MTBE, Across) using liquid-liquid extraction. The combined MTBE fractions were dried under a gentle nitrogen gas flow. Then the dried sample was reconstituted with 120 μl of 50% methanol [methanol/water (v/v)]. The reconstituted sample was divided in two fractions, one for estrogen and one for androgen analyses
Estrogen analysis An ultra-high pressure liquid chromatography (NEXERA X2, Shimadzu Corporation) system with a C18 column (Infinity Lab Poroshell 120 EC-C18 column, 4.6×75 mm, 2.7 μm, Agilent) and gradient was used to separate estrogens in one of the prepared fractions. The separated estrogens were selected and quantified by mass spectrometry (Qtrap 5500, AB Sciex) by using multiple reaction monitoring (MRM) mode in negative ion ESI.
Androgen analysis The other prepared fraction was injected onto the UPLC system, and the androgens were separated on a C18 column (Zorbax Eclipse Plus C18 column, 150 mm×2.1 mm, 3.5 μm, Agilent). A gradient was used. The separated androgens were quantified on the Qtrap 5500 mass spectrometer using the MRM mode in positive ion ESI.

MultiQuant Software (version 3.0.3, AB Sciex) was used for the data acquisition and quantification for estrogens and androgens.

### RNA-Seq Analysis

Tumor RNA was extracted from the mice used in the C4-2 zanubrutinib treatment experiment, 4 samples from each group. RNA was extracted with GenElute Mammalian Total RNA miniprep kit (Sigma-Aldrich). The Case Western Reserve University Genomics Core performed the RNA-seq using the HumanHT-12 v4 Expression BeadChip and iScan (Illumina). Hybrid signals were analyzed with Illumina GenomeStudio Software 2011.1 and normalized to the vehicle control group. Heatmaps were generated with HemI software (version 1.0). GSEA was used to correlate the 5α-Abi expression data with an androgen receptor-selective gene set described elsewhere (Arora et al., 2013). The GSEA enrichment plot was generated as described elsewhere (Subramanian et al., 2005).

### Human Tissue Studies

All deidentified human tissues were obtained at the Cleveland Clinic under institutional review board-approved protocols. Fresh prostate tissue cores (60 to 100 mg) from 42 patients were obtained for germline DNA analysis and DHEA metabolism.
DHEA metabolism studies: Forty to 60 mg tissue was used for DHEA metabolism detection. Briefly, tissue cores were minced and aliquoted in 2 equal portions. One was treated with zanubrutinib, and the other was treated with DMSO. Both tissues were maintained in 3 ml DMEM containing 10% fetal bovine serum and incubated in a 5% CO₂ humidified incubator. After 12 hours of culture, [³H]-DHEA was added to each portion. Cell culture medium was collected at the indicated time points, and HPLC was performed as described above. Protein was extracted from about 20 mg human prostate tissue, followed by 3βHSD1 immunoprecipitation and western blot. To determine the effects of BMX inhibition, the remaining 20-40 mg tissue was minced and aliquoted in 2 equal parts. One portion was treated with zanubrutinib, and the other was treated with DMSO. Both tissues were maintained in 3 ml DMEM containing 10% fetal bovine serum and incubated in a 5% CO2 humidified incubator. After 12 hours of culture, 10 nM DHEA was added to each portion. Seven days later, tissue was homogenized with ice-cold radioimmunoprecipitation assay (RIPA) lysis buffer (Sigma-Aldrich) containing protease inhibitors (Roche) and phosphatase inhibitors (Sigma-Aldrich) using a homogenizer to extract protein; immunoprecipitation and western blot were then performed.
Genotyping studies: A total of 42 clinical prostate tissues were obtained. Germline DNA was genotyped for HSD3B1 as described previously (Hearn et al., 2016), and 19, 18 and 5 cases had 0, 1 and 2 copies of the adrenal-permissive HSD3B1(1245C) allele, respectively. Of these, 0/19, 4/18 and 3/5 were observed to have DHEA metabolism, and the 7 showing metabolism were included to assess effects of zanubritinib on 3βHSD1 metabolic activity.

### Statistical Analysis

Statistical data analyses were performed in GraphPad Prism software (version 9.0.0) and Microsoft Excel (version 16.43). In general, for mouse xenograft studies, progression-free survival was determined by Kaplan-Meier analysis followed by a log-rank test to compare among groups. For other comparative analyses, an unpaired two-tailed/test was used unless otherwise noted. P<0.05 was considered statistically significant.

### Results

### Phosphorylation of 3βHSD1 Y344 is Required for DHEA Metabolism

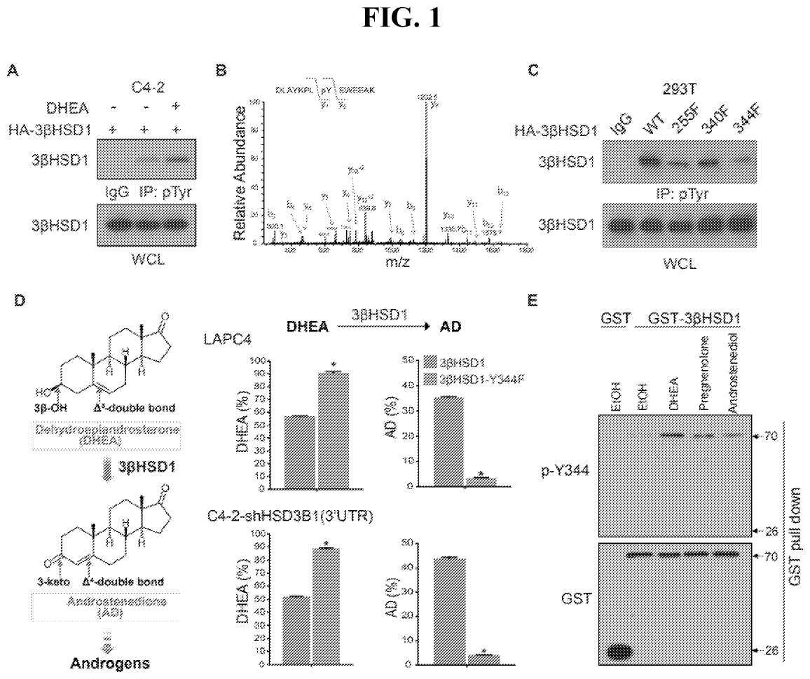

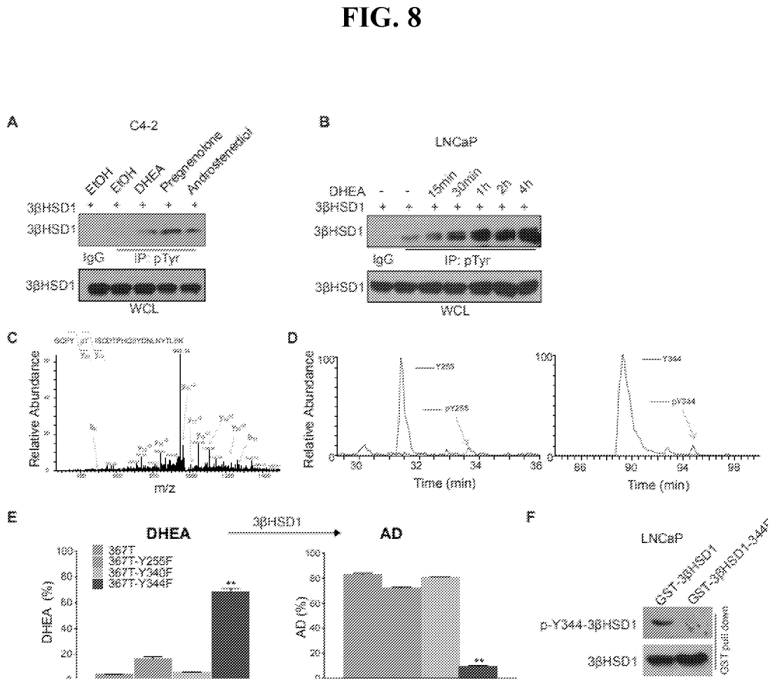

To test whether phosphorylation is necessary for 3βHSD1 activity, we overexpressed HA-3βHSD1 in C4-2 cells. Immunoblotting analyses of immunoprecipitated HA-3βHSD1 with anti-phospho-tyrosine antibodies showed that 3βHSD1 undergoes tyrosine phosphorylation, which is induced in the presence of its substrates DHEA, pregnenolone, and androstenediol (FIGS. 1A and 8A-8B). Mass spectrometry analyses of immunoprecipitated GST-3βHSD1 expressed in C4-2 cells showed that 3βHSD1 was phosphorylated at tyrosine (Y) 255 and tyrosine (Y) 344 (FIGS. 1B and 8C-8D). This result was further supported by immunoblotting analyses. Mutation of Y344 or Y255 to phenylalanine (F) reduced tyrosine phosphorylation of 3βHSD1 (FIG. 1C). Further, we found that phosphorylation of Y344 but not Y255 is required for 3βHSD1 cellular activity because DHEA metabolism was significantly reduced after mutation to phenylalanine (FIGS. 1D and 8E). To gain further insight about the mechanisms of this modification, we custom-designed an anti-phospho-3βHSD1 Y344 (anti-3βHSD1 pY344) antibody. After determining that it had excellent specificity (FIG. 8F), we used it to detect 3βHSD1 PY344 in C4-2 and LNCaP cells. We found that 3βHSD1 pY344 was induced by DHEA treatment (FIG. 1E). Collectively, these results indicate that Y344 phosphorylation is essential for 3βHSD1 enzyme activity in cells.

### BMX is Required for DHEA Metabolism by 3βHSD1

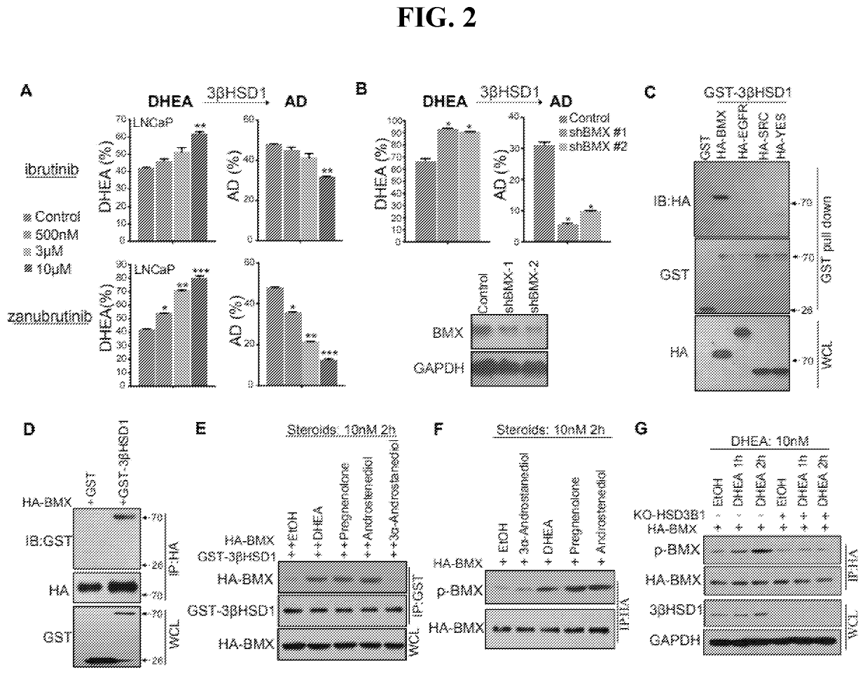

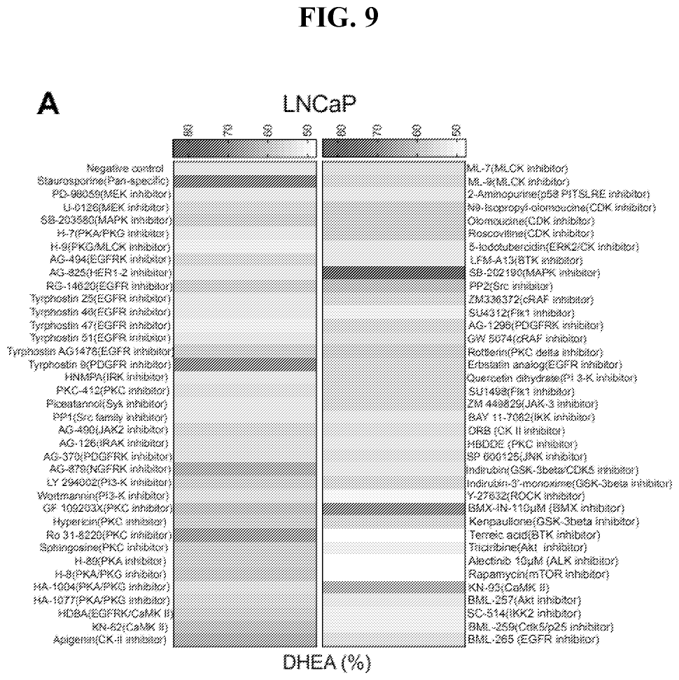

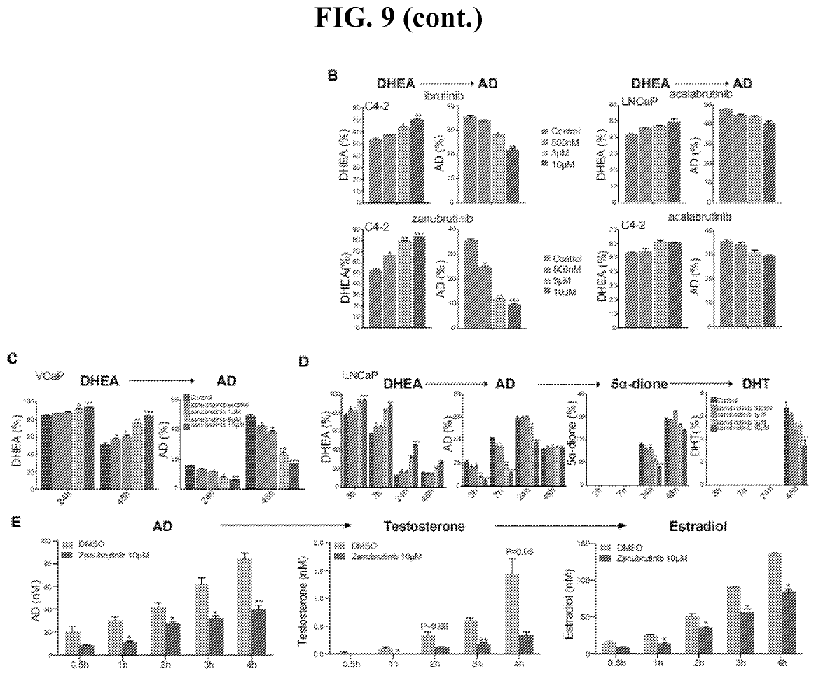

To identify the kinase that phosphorylates 3βHSD1, we used a kinase inhibitor library to screen in an unbiased fashion for pharmacologic inhibitors that block [³H]-DHEA metabolism (FIG. 9A). HPLC analyses showed that pharmacologic inhibitors of BMX or PDGFR block [³H]-DHEA metabolism to AD. Kinase prediction based on Y344 using the phosphoNET Kinase Predictor (http://www.phosphonet.ca/kinasepredictor.aspx?uni=P14060&ps=Y344) showed that BMX had the greatest potential to phosphorylate 3βHSD1 at Y344. Further, we tested several BTK/BMX inhibitors, with HPLC analyses showing that zanubrutinib or ibrutinib treatment significantly decreased DHEA metabolism in multiple prostate cancer (LNCaP, C4-2 and VCaP) cell lines (FIGS. 2A and 9B-9D). The 3β-OH→3-keto and Δ⁵→Δ⁴ reactions catalyzed by 3βHSD1 to synthesize AD does not only serve to make this a substrate of steroid-5α-reductase (SRD5A) (Chang et al., 2011; Russell and Wilson, 1994)—these reactions are also essential to generate 3-keto, Δ⁴-steroid substrates (e.g., AD and testosterone) for aromatase and estrogen biosynthesis (Simpson et al., 2002). Thus, we examined the effect of BMX inhibition on estrogen synthesis from DHEA as a precursor. We found that zanubrutinib also inhibits the production of estradiol from DHEA (FIG. 9E).

We generated LNCaP cells stably expressing shNT (non-targeting control) or shRNA sequences against BMX (shBMX) and found that knocking down BMX also inhibited DHEA metabolism (FIG. 2B). We next examined the interaction between 3βHSD1 and BMX. Co-immunoprecipitation assays showed that 3βHSD1 interacted with BMX (FIGS. 2C and 2D), and this interaction was induced by the 3βHSD1 substrates DHEA, pregnenolone, and androstenediol (FIG. 2E). Moreover, we found that 3βHSD1 substrates induced the phosphorylation of BMX (FIG. 2F) and that 3βHSD1 substrates are needed for the induction of BMX phosphorylation (FIG. 2G). In the absence of 3βHSD1, DHEA failed to activate the phosphorylation of BMX, suggesting that DHEA-mediated 3βHSD1-BMX interaction is needed for BMX activation. Taken together, our findings indicate that BMX is required for DHEA metabolism by 3βHSD1.

### BMX Directly Phosphorylates 3βHSD1 at Y344

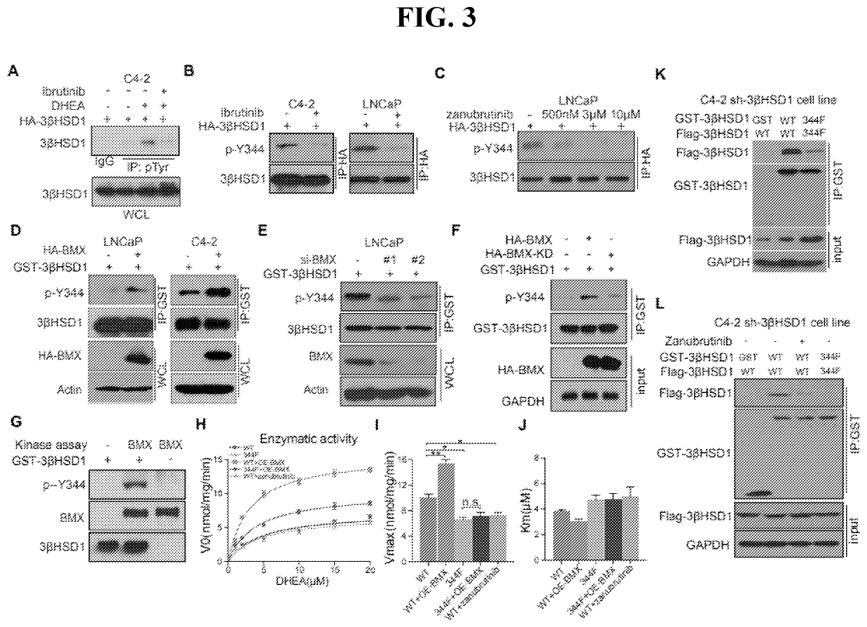

To investigate the role of BMX in phosphorylation of 3βHSD1, we inhibited BMX activity in C4-2 and LNCaP cells. Immunoblotting analyses of immunoprecipitated HA-3βHSD1 with anti-phospho-tyrosine and anti-3βHSD1 pY344 antibodies showed that the BMX inhibitors ibrutinib and zanubrutinib blocked DHEA-induced phosphorylation of 3βHSD1 (FIGS. 3A-3C). We overexpressed HA-BMX in C4-2 and LNCaP cells. Immunoblotting analyses of immunoprecipitated HA-3βHSD1 with anti-3βHSD1 pY344 antibodies showed BMX enhanced phosphorylation of 3βHSD1 PY344 (FIG. 3D). Moreover, knockdown of BMX substantially reduced 3βHSD1 pY344 phosphorylation with DHEA stimulation in LNCaP cells (FIG. 3E). Furthermore, overexpression of a kinase-dead BMX mutant (BMX-KD) in C4-2 cells strikingly failed to stimulate 3βHSD1 PY344 phosphorylation that was induced by WT-BMX (FIG. 3F). To determine whether BMX directly phosphorylates 3βHSD1, we carried out in vitro kinase assays by mixing purified GST-3βHSD1 and HA-BMX. The assays showed that BMX directly phosphorylated 3βHSD1 at Y344 (FIG. 3G). These results indicate that BMX is the kinase that directly phosphorylates 3βHSD1 at Y344.

3βHSD1 pY344 Enhances Dimerization

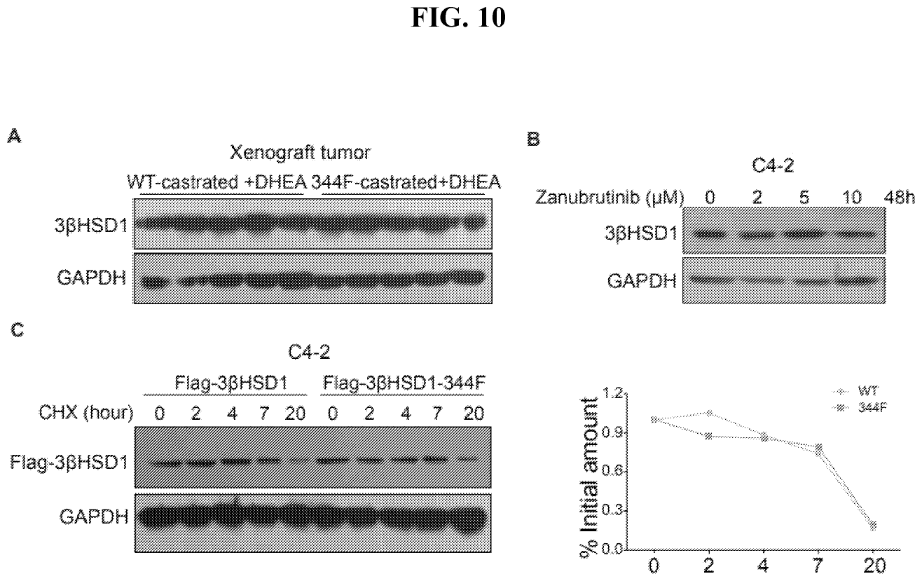

We next investigated how Y344 phosphorylation promotes 3βHSD1 cellular enzymatic activity. We purified 3βHSD1 or 3βHSD1-Y344F mutant and performed an NAD+ turnover assay in vitro. The results showed that WT enzyme purified from zanubrutinib-treated cells and the phospho-mutant of 3βHSD1 had lower catalytic activity (Vmax); WT enzyme purified from cells with overexpressed BMX had a marginal increase in catalytic activity (Vmax), and no significant increase in Vmax was observed for 3βHSD1 phospho-mutant expressed in cells with overexpressed BMX (FIG. 3H, I). 3βHSD1 phosphorylation did not significantly affect the Michaelis constant (Km) of DHEA (FIG. 3J). These results suggest that in a purified in vitro context, phosphorylation appears to have minimal influence on observed enzymatic activity. Furthermore, phosphorylation of Y344 at 3βHSD1 had no effect on its protein expression or degradation (FIGS. 10A and 10C), nor did zanubrutinib affect the level of 3βHSD1 protein expression (FIG. 10B). Some hydroxysteroid dehydrogenase enzymes are known to exist as dimers (Gomez-Sanchez et al., 2001). To further investigate how Y344 phosphorylation may influence 3βHSD1 activity, we tested whether Y344 phosphorylation regulates 3βHSD1 dimer formation in C4-2 cells co-transfected with Flag-3βHSD1 and GST-3βHSD1 and treated with DHEA. Co-immunoprecipitation of Flag-3βHSD1 and GST-3βHSD1 was inhibited by blocking Y-phosphorylation using the Y344F mutation, as well as with zanubrutinib treatment, suggesting that 3βHSD1 pY344 enhances dimerization in cells (FIGS. 3K and 3L). These results suggest that phosphorylation may promote cellular dimerization and thus enhance enzymatic activity of 3βHSD1 in a cellular context.

### BMX Blockade and Inhibition of 3βHSD1 Phosphorylation Impedes Expression of Androgen-Regulated Genes and Prostate Cancer Proliferation

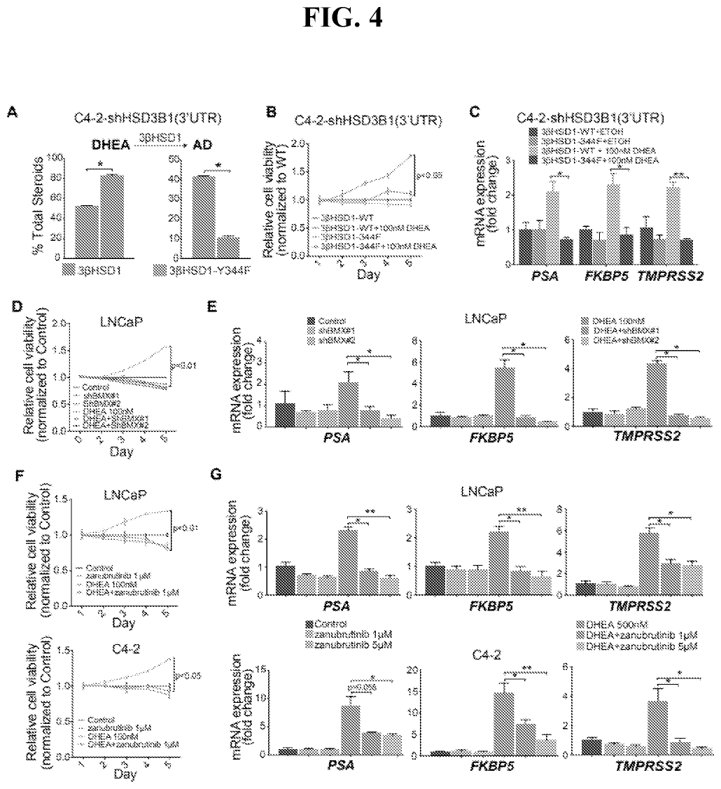

We next investigated the biological function of 3βHSD1 pY344. To determine whether phosphorylation of 3βHSD1 affects prostate cancer cell growth, we produced C4-2 cell lines that stably expressed 3βHSD1 (WT) or 3βHSD1-Y344F. DHEA metabolism was retarded in the 3βHSD1-Y344F cell line (FIG. 4A). We then tested cell viability and proliferation using the WST-1 assay and found that the Y344F mutation of 3βHSD1 inhibits DHEA-stimulated cell proliferation (FIG. 4B). Further, the 3βHSD1 Y344F mutation inhibited AR-dependent transcriptional activity of canonical AR-regulated genes (FIG. 4C). Our results suggest that 3βHSD1 pY344 phosphorylation promotes prostate cancer cell progression.

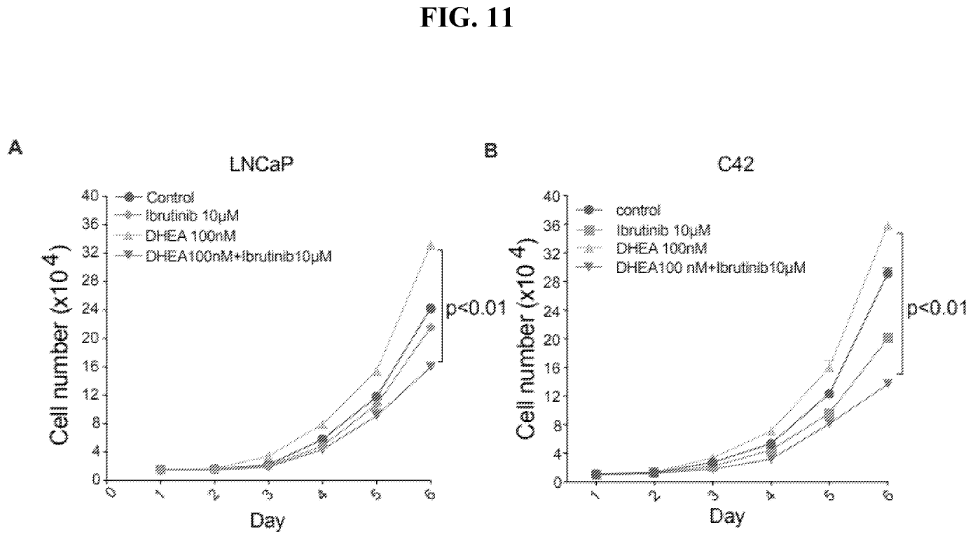

To explore whether targeting regulatory kinases can inhibit prostate cancer cell proliferation, we generated LNCaP cells stably expressing shNT (non-targeting control) or two shRNA sequences against BMX. After overnight serum deprivation, we assessed proliferation in the presence or absence of DHEA. The results indicated that knocking down BMX inhibited cell proliferation that is induced by DHEA (FIG. 4D). In addition, qPCR results showed that knocking down BMX reduced DHEA-induced AR transcriptional activity (FIG. 4E). Based on these findings, we hypothesized that BMX inhibitors also inhibit prostate cancer proliferative activity. We treated LNCaP and C4-2 cells with zanubrutinib and detected cell proliferation and AR target gene expression. Our results demonstrated that zanubrutinib effectively inhibited proliferation (FIG. 4F) and AR transcriptional activity in the presence of DHEA (FIG. 4G) in LNCaP and C4-2 cells. We also treated LNCaP and C-42 cells with another BMX inhibitor, ibrutinib, and assessed viability using Trypan blue staining (FIGS. 11A and 11B), the results of which were consistent with the cell proliferation results. In conclusion, our findings suggest that the phosphorylation of 3βHSD1 Y344 promotes prostate cancer proliferation, and targeting BMX as its regulatory kinase blocks the growth of prostate cancer cells.

### 3βHSD1-Y344F Blocks CRPC Growth In Vivo

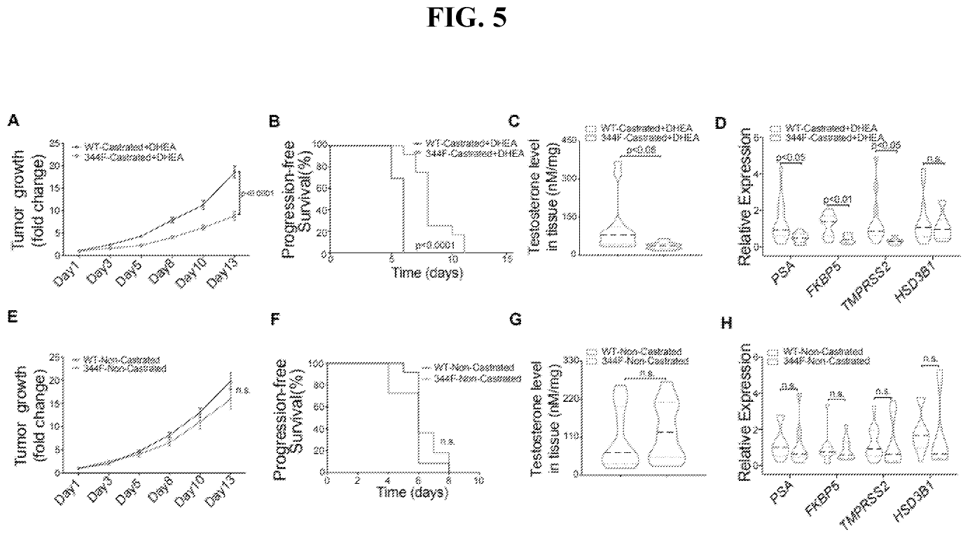

We next sought to determine the requirement for 3βHSD1 pY344 phosphorylation in development of CRPC in vivo in mouse xenograft models. We generated C4-2 cell lines that stably expressed 3βHSD1 (WT) or 3βHSD1-Y344F. subcutaneously injected the cells into NSG male mice to develop tumors, followed by surgical castration and subcutaneous implantation of 90-day sustained-release DHEA pellets to mimic the human physiology that occurs with ADT and CRPC development, similar to our prior studies (Li et al., 2021; Li, 2016; Li et al., 2015; Zhu et al., 2018). We found that the Y344F mutation of 3βHSD1 inhibited DHEA-induced tumor growth and prolonged progression-free survival in C4-2 xenograft models of CRPC (FIGS. 5A and 5B). Mass spectrometry assessment of androgens in xenograft tissues (FIG. 5C) demonstrated that the Y344F mutation inhibited tumor growth through loss of intratumoral androgen production. Reduction in AR transcriptional activity also was detected in xenograft tumors carrying mutated Y344F (FIG. 5D). In contrast, the Y344F mutation had no significant effect on growth, tissue testosterone, or androgen signaling in xenografts growing in eugonadal mice (FIG. 5E-5H).

### Pharmacologic BMX Blockade Inhibits Androgen Biosynthesis and CRPC Growth In Vivo

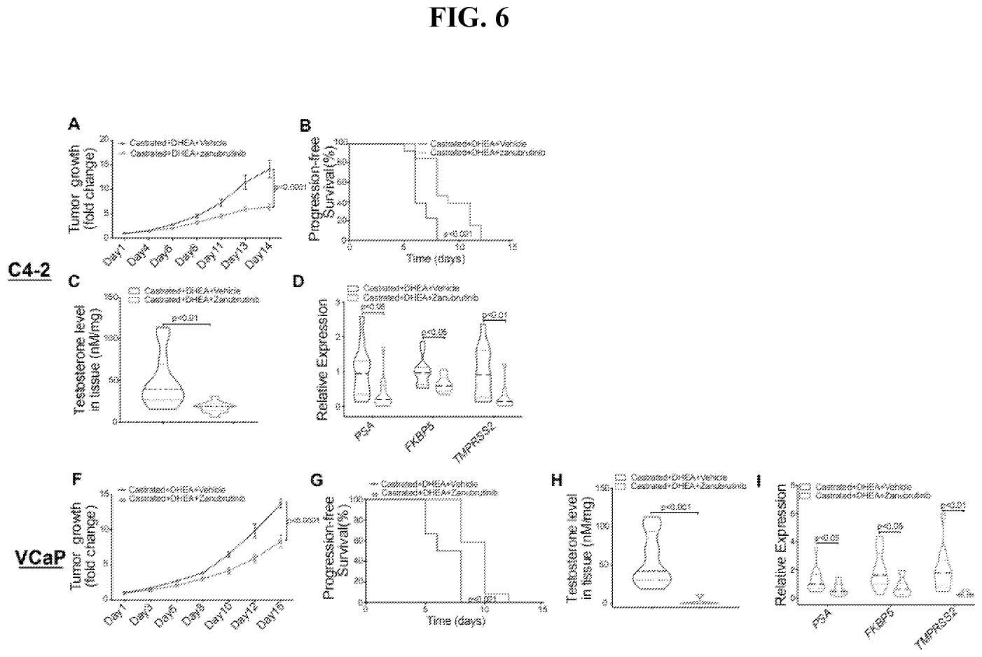

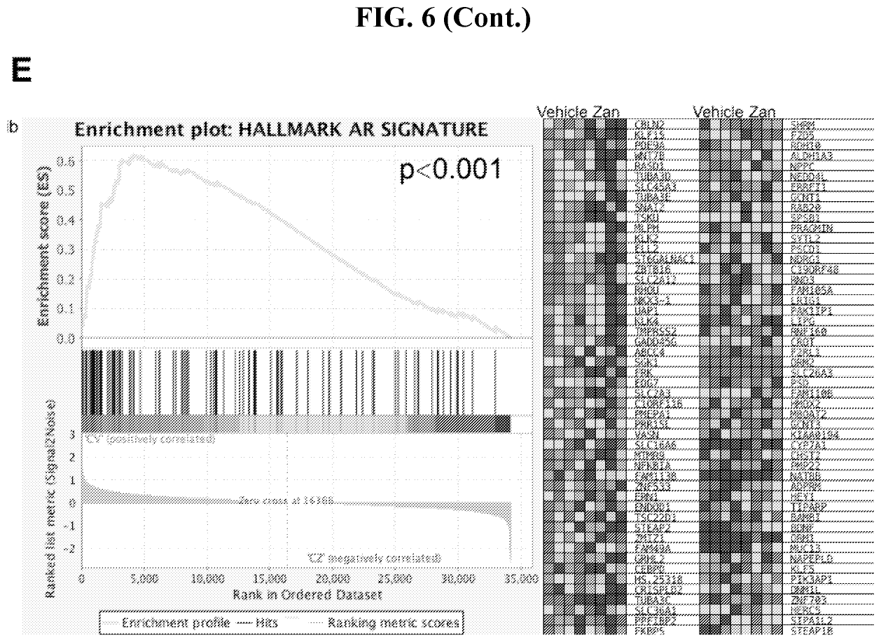

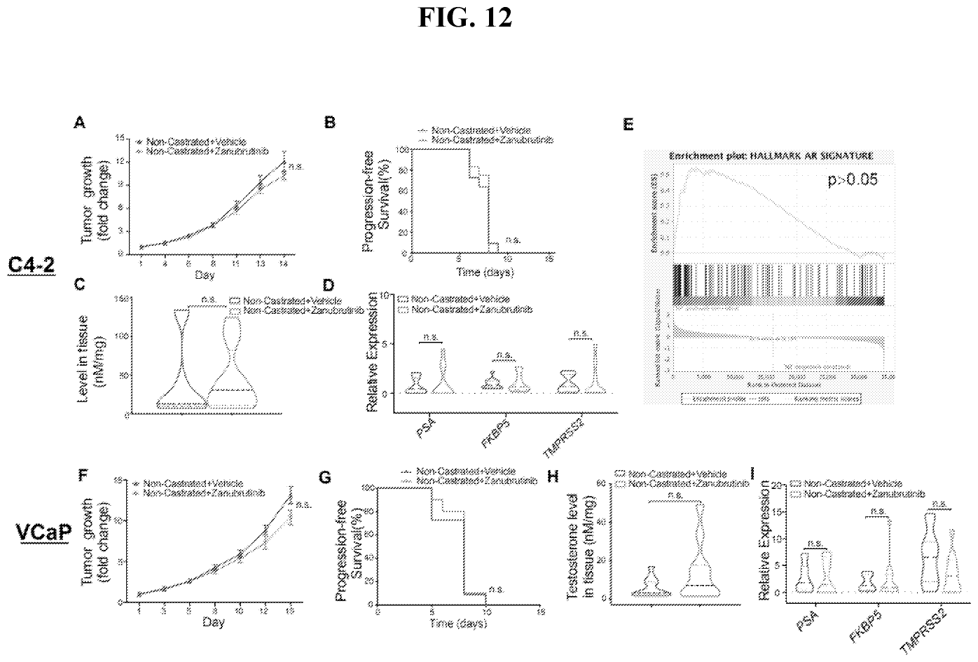

To investigate whether targeting phosphorylation blocks prostate cancer growth in vivo, we determined how effectively zanubrutinib blocked CRPC growth using mouse xenograft models. We established C4-2 or VCaP CRPC tumors with castration and DHEA pellet implantation. The mice were treated with zanubrutinib at a dose of 15 mg/kg or vehicle by oral gavage twice daily. Treatment with zanubrutinib led to significant tumor growth inhibition in both models compared to vehicle control (P<0.0001) (FIGS. 6A and 6F). In contrast, zanubrutinib had virtually no effect on growth and androgen signaling in xenografts growing in eugonadal mice (FIGS. 12A and 12F). Differences in progression-free survival were similarly significant for zanubrutinib treatment in mice with CRPC (FIGS. 6B and 6G; P<0.0001) but not xenografts grown in the eugonadal mice (FIGS. 12B and 12G). Tumors from zanubrutinib-treated mice with CRPC displayed lower androgen production and AR target gene (PSA, FKBP5, TMPRS9) expression (FIGS. 6C-6D and 6H-I). By contrast, zanubrutinib had no significant effect on untreated tumors (FIGS. 12C-12D and 12H-12I). Unbiased RNA-Seq and Gene Set Enrichment Analysis (GSEA) similarly demonstrated that zanubrutinib inhibited AR-regulated genes (FIG. 6E). Zanubrutinib had no significant effect on AR regulation in eugonadal tumors (FIG. 12E).

### Effects of BMX Inhibition on 3βHSD Phosphorylation and Androgen Synthesis in Fresh Human Prostate Tissues

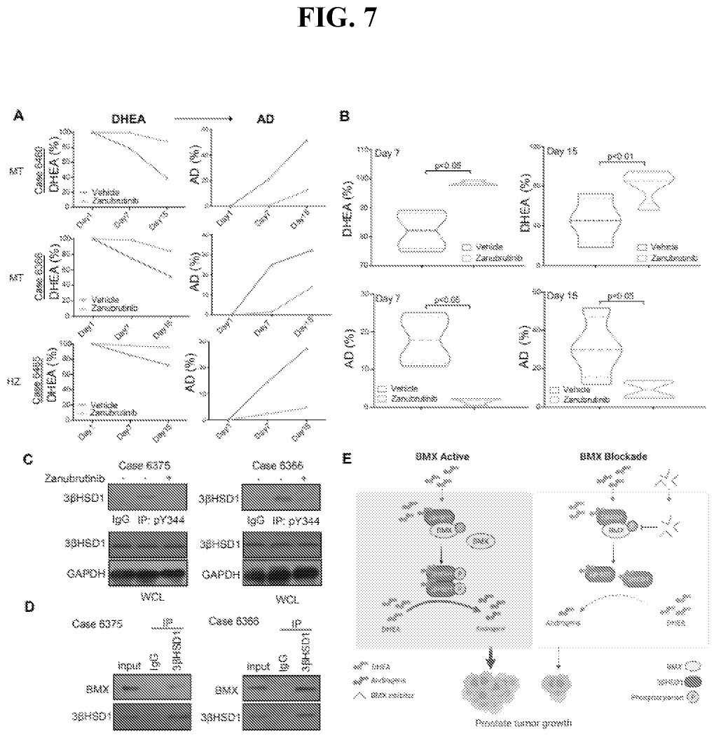

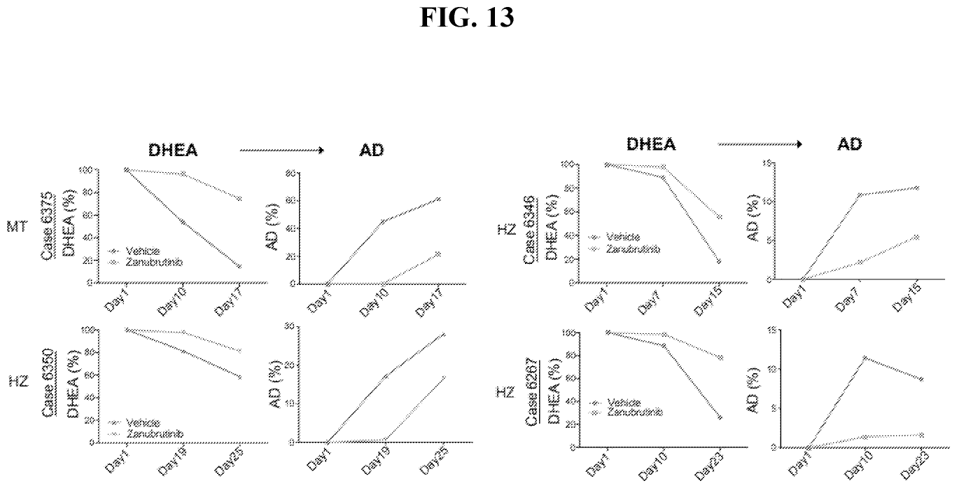

We next investigated the effects of BMX pharmacologic blockade in fresh prostate tissues cultured for metabolic assessment from men undergoing radical prostatectomy. Tissues were treated with [³H]-DHEA with or without zanubrutinib, and HPLC was performed on steroids extracted from medium to detect metabolism to steroids downstream of 3βHSD1. Tissues from a total of 7 patients, all of whom harbored the adrenal-permissive HSD3B1 allele (3 homozygous and 4 heterozygous) had detectable DHEA metabolism and thus were assessable for reversibility with zanubrutinib. Notably, our results showed that the metabolism of DHEA was inhibited by zanubrutinib in all 7 prostate tissues (FIGS. 7A-7B and 13). Immunoprecipitation and western blot results showed that both phosphorylation of 3βHSD1 and metabolic flux from DHEA to AD were reduced by inhibiting BMX with zanubrutinib (FIG. 7C). In addition, the interaction between BMX and 3βHSD1 was observed in human prostate tissues (FIG. 7D). Taken together, these results show the potential therapeutic effects of targeting 3βHSD1 phosphorylation using BMX inhibitors for the treatment of men with CRPC (FIG. 7E) and more generally, the role of BMX in physiologic regulation of extragonadal sex steroid biosynthesis.

Androgen dependence is a major hallmark of prostate cancer, even after progression on hormonal therapy (Centenera et al., 2018; Watson et al., 2015). Clinical responses to ADT are almost always followed by development of CRPC that occurs in major part because tumors engage metabolic mechanisms to make their own potent androgens from extragonadal precursor steroids (Attard et al., 2016; Dai et al., 2017). In the absence of gonadal testosterone, as is the case with ADT, the adrenals are the major source of precursors for sex steroids, and HSD3B1 encodes for the peripherally expressed enzyme that is necessary for conversion from DHEA to biologically active androgens and estrogens (Labrie, 2004; Naelitz and Sharifi, 2020; Sabharwal and Sharifi, 2019). Genetic evidence from at least 10 prostate cancer cohorts on inheritance of the adrenal-permissive HSD3B1(1245C) allele demonstrates that increased metabolic flux through 3βHSD1 hastens androgen biosynthesis, progression to CRPC, and prostate cancer mortality (Agarwal et al., 2017; Hearn et al., 2020; Hettel and Sharifi, 2018; Ku et al., 2019; Prizment et al., 2021; Thomas and Sharifi, 2020). Importantly, 3βHSD1 catalyzes steroid Δ⁵→Δ⁴ isomerization and 3β-OH→3-keto oxidation-reactions that are absolutely required for all pathways from the starting structure of (Δ⁵, 3β-OH) cholesterol or adrenal DHEA to testosterone and DHT (Sharifi, 2013). This may explain why treatment with abiraterone or enzalutamide does not overcome the adverse clinical outcomes conferred by the sustained androgen biosynthesis of adrenal-permissive HSD3B1 inheritance as determined in studies from 4 institutions and over 800 men (Khalaf et al., 2020; Lu et al., 2020; Sharifi, 2020). Together, the existing clinical data suggest that direct inhibition of 3βHSD1 is necessary as a pharmacologic maneuver. However, until now, there has been no clinically appropriate mechanistic strategy to effectively pharmacologically block 3βHSD1 and to reverse the adverse clinical biology of the adrenal-permissive form of 3βHSD1 that is inherited by about half of all men with prostate cancer.

It is believed that this Example is the first to identify a posttranslational modification that is necessary for 3βHSD1 activity in cells. The BMX kinase is known to be up-regulated with ADT (Chen et al., 2018). However, the mechanisms downstream of BMX, effects on tumor metabolism, and the context for contributions to CRPC have remained elusive. This Example establishes an essential role for BMX in regulating extragonadal sex steroid biosynthesis and suggest that the adverse clinical biology and poor survival in men with adrenal-permissive HSD3B1 inheritance can be directly reversed by inhibiting BMX. Because they are in the same family of TEC non-receptor kinases, there is a major overlap between tyrosine kinase inhibitors of BTK and BMX (Seixas et al., 2020); Tamagnone et al., 1994). Thus, largely attributable to clinical advances in B-cell leukemias for the purpose of BTK inhibition, several options for BTK/BMX inhibitors are available for clinical trials, including, for example, ibrutinib, zanubrutinib, alacabrutinib and abivertinib (Bond and Woyach, 2019: Burger et al., 2015).

Based on our mechanistic findings here, a multi-institutional phase 2 clinical trial of abivertinib plus abiraterone could be conducted for men with metastatic CRPC who inherit the adrenal-permissive HSD3B1 allele. This trial would test an entirely new mechanistic concept for the treatment of metastatic CRPC, in a disease and treatment space where use of kinase inhibitors have not previously been shown to be effective (Araujo et al., 2013). Unlike other solid tumors, such as melanoma and lung cancer where kinase inhibitors have been successful, activating kinase mutations that are bona fide genetic drivers are generally uncommon in human prostate cancer. Instead, in this instance, kinase activity is required for the adrenal-permissive 3βHSD1 enzyme, which is directly and mechanistically linked to treatment resistance and prostate cancer lethality in multiple human cohorts.

Finally, 3βHSD1 also lies one step upstream of aromatase, which is required for the generation of estrogens. The Δ⁴. 3-keto-steroid products of 3βHSD1, AD and testosterone, are converted to estrone and estradiol, respectively (Chumsri et al., 2011; Simpson et al., 2002). Similar to the context of men absent gonadal testosterone, emerging evidence suggests an essential role for HSD3B1 inheritance in postmenopausal women who have only adrenal precursors as their physiologic source of sex steroids. The frequency of the homozygous adrenal-permissive HSD3B1 genotype is enriched in women with estrogen-driven postmenopausal breast cancer and occurs in about 15% of these tumors (Kruse et al., 2021). Furthermore, these women with homozygous adrenal-permissive HSD3B1 inheritance have a significantly increased rate of metastatic recurrence after treatment for localized breast cancer, even with hormonal therapy (Flanagan, 2022), suggesting that these women have more aggressive disease and that new strategies are required to improve clinical outcomes.

### REFERENCES

Abida, et al. (2019). Genomic correlates of clinical outcome in advanced prostate cancer. Proc Natl Acad Sci USA 116, 11428-11436.
Agarwal, et al. (2017). Independent Validation of Effect of HSD3B1 Genotype on Response to Androgen-Deprivation Therapy in Prostate Cancer. JAMA Oncol.
Araujo, et al. (2013). Docetaxel and dasatinib or placebo in men with metastatic castration-resistant prostate cancer (READY): a randomised, double-blind phase 3 trial. Lancet Oncol 14, 1307-1316.
Arora, et al. (2013). Glucocorticoid receptor confers resistance to antiandrogens by bypassing androgen receptor blockade. Cell 155, 1309-1322.
Attard, (2016). Prostate cancer. Lancet 387, 70-82.
Auchus, R. J., and Sharifi, N. (2020). Sex Hormones and Prostate Cancer. Annu Rev Med 71, 33-45.
Bond, D. A., and Woyach, J. A. (2019). Targeting BTK in CLL: Beyond Ibrutinib. Curr Hematol Malig Rep 14, 197-205.
Burger, et al. (2015). Ibrutinib as Initial Therapy for Patients with Chronic Lymphocytic Leukemia. N Engl J Med 373, 2425-2437.
Centenera, et al., (2018). New Opportunities for Targeting the Androgen Receptor in Prostate Cancer. Cold Spring Harb Perspect Med 8.
Chang, et al. (2013). A gain-of-function mutation in DHT synthesis in castration-resistant prostate cancer. Cell 154, 1074-1084.
Chang, et al., (2011). Dihydrotestosterone synthesis bypasses testosterone to drive castration-resistant prostate cancer. Proc Natl Acad Sci USA 108, 13728-13733.
Chen, et al., (2018). BMX-Mediated Regulation of Multiple Tyrosine Kinases Contributes to Castration Resistance in Prostate Cancer. Cancer Res 78, 5203-5215.
Chumsri, et al., (2011). Aromatase, aromatase inhibitors, and breast cancer. J Steroid Biochem Mol Biol 125, 13-22.
Dai, et al. (2010). Compensatory upregulation of tyrosine kinase Etk/BMX in response to androgen deprivation promotes castration-resistant growth of prostate cancer cells. Cancer Res 70, 5587-5596.
Dai, C., Heemers, H., and Sharifi, N. (2017). Androgen Signaling in Prostate Cancer. Cold Spring Harb Perspect Med 7.
Einstein, D. J., Arai, S., and Balk, S. P. (2019). Targeting the androgen receptor and overcoming resistance in prostate cancer. Curr Opin Oncol 31, 175-182.
Evaul, et al., (2010). 3beta-hydroxysteroid dehydrogenase is a possible pharmacological target in the treatment of castration-resistant prostate cancer. Endocrinology 151, 3514-3520.
Flanagan, et al., (2022). Association of HSD3B1 genotype and clinical outcomes in postmenopausal estrogen-receptor positive breast cancer. In Society for Surgical Oncology (Dallas, TX).
Gomez-Sanchez et al., (2001). The 11beta hydroxysteroid dehydrogenase 2 exists as an inactive dimer. Steroids 66, 845-848.
Hearn, et al. (2016). HSD3B1 and resistance to androgen-deprivation therapy in prostate cancer: a retrospective, multicohort study. Lancet Oncol 17, 1435-1444.
Hearn, et al. (2020). HSD3B1 Genotype and Clinical Outcomes in Metastatic Castration-Sensitive Prostate Cancer. JAMA Oncol 6, e196496.
Hettel, D., and Sharifi, N. (2018). HSD3B1 status as a biomarker of androgen deprivation resistance and implications for prostate cancer. Nat Rev Urol 15, 191-196.
Khalaf, et al. (2020). HSD3B1 (1245A>C) germline variant and clinical outcomes in metastatic castration-resistant prostate cancer patients treated with abiraterone and enzalutamide: results from two prospective studies. Ann Oncol 31, 1186-1197.
Kruse et al. (2021). Adrenal-permissive HSD3B1 genetic inheritance and risk of estrogen-driven postmenopausal breast cancer. JCI Insight 6.
Ku, S. Y., Gleave, M. E., and Beltran, H. (2019). Towards precision oncology in advanced prostate cancer. Nat Rev Urol 16, 645-654.
Labrie, F. (2004). Adrenal androgens and intracrinology. Semin Reprod Med 22, 299-309.
Li, et al. (2021). Hexose-6-phosphate dehydrogenase blockade reverses prostate cancer drug resistance in xenograft models by glucocorticoid inactivation. Sci Transl Med 13.
Li, et al., (2016). Redirecting abiraterone metabolism to biochemically fine tune prostate cancer anti-androgen therapy. Nature 533, 547-551.
Li, et al., (2015). Conversion of abiraterone to D4A drives anti-tumour activity in prostate cancer. Nature 523, 347-351.
Lorence, et al., (1990). Human 3 beta-hydroxysteroid dehydrogenase/delta 5----4isomerase from placenta: expression in nonsteroidogenic cells of a protein that catalyzes the dehydrogenation/isomerization of C21 and C19 steroids. Endocrinology 126, 2493-2498.
Lu, C, et al. (2020). Treatment with abiraterone and enzalutamide does not overcome poor outcome from metastatic castration-resistant prostate cancer in men with the germline homozygous HSD3B1 c. 1245C genotype. Ann Oncol 31, 1178-1185.
Miller, W. L., and Auchus, R. J. (2011). The molecular biology, biochemistry, and physiology of human steroidogenesis and its disorders. Endocr Rev 32, 81-151.
Miyamoto, et al. (2012). Androgen receptor signaling in circulating tumor cells as a marker of hormonally responsive prostate cancer. Cancer Discov 2, 995-1003.
Mostaghel, et al. (2021). Circulating and Intratumoral Adrenal Androgens Correlate with Response to Abiraterone in Men with Castration-Resistant Prostate Cancer. Clin Cancer Res 27, 6001-6011.
Naelitz, B. D., and Sharifi, N. (2020). Through the Looking-Glass: Reevaluating DHEA Metabolism Through HSD3B1 Genetics. Trends Endocrinol Metab 31, 680-690.
Prizment, et al., (2021). Prostate Cancer Mortality Associated with Aggregate Polymorphisms in Androgen-Regulating Genes: The Atherosclerosis Risk in the Communities (ARIC) Study. Cancers (Basel) 13.
Russell, D. W., and Wilson, J. D. (1994). Steroid 5 alpha-reductase: two genes/two enzymes. Annu Rev Biochem 63, 25-61.
Sabharwal, N., and Sharifi, N. (2019). HSD3B1 Genotypes Conferring Adrenal-Restrictive and Adrenal-Permissive Phenotypes in Prostate Cancer and Beyond. Endocrinology 160, 2180-2188.
Sanjana, N. E., Shalem, O., and Zhang, F. (2014). Improved vectors and genome-wide libraries for CRISPR screening. Nat Methods 11, 783-784.

Seixas, et al. (2020). Structural and biophysical insights into the mode of covalent binding of rationally designed potent BMX inhibitors. RSC Chem Biol 1, 251-262.
Sharifi, N. (2013). Minireview: Androgen metabolism in castration-resistant prostate cancer. Mol Endocrinol 27, 708-714.
Sharifi, N. (2020). Homozygous HSD3B1(1245C) inheritance and poor outcomes in metastatic castration-resistant prostate cancer with abiraterone or enzalutamide: what does it mean? Ann Oncol 31, 1103-1105.
Sharifi, N., McPhaul, M. J., and Auchus, R. J. (2010). “Getting from here to there”—mechanisms and limitations to the activation of the androgen receptor in castration-resistant prostate cancer. J Investig Med 58, 938-944.
Simpson, et al., (2002). Aromatase—a brief overview. Annu Rev Physiol 64, 93-127. Subramanian, A., Tamayo, P., Mootha, V. K., Mukherjee, S., Ebert, B. L., Gillette, M. A.,
Paulovich, et al. (2005). Gene set enrichment analysis: a knowledge-based approach for interpreting genome-wide expression profiles. Proc Natl Acad Sci USA 102, 15545-15550.
Tamagnone, et al., (1994). BMX, a novel nonreceptor tyrosine kinase gene of the BTK/ITK/TEC/TXK family located in chromosome Xp22.2. Oncogene 9, 3683-3688.
Thomas, et al., (2002). Structure/function relationships responsible for the kinetic differences between human type 1 and type 2 3beta-hydroxysteroid dehydrogenase and for the catalysis of the type 1 activity. J Biol Chem 277, 42795-42801.
   Thomas, L., and Sharifi, N. (2020). Germline HSD3B1 Genetics and Prostate Cancer Outcomes. Urology 145, 13-21.
Waitkus, et al., (2014). Signal integration and gene induction by a functionally distinct STAT3 phosphoform. Mol Cell Biol 34, 1800-1811.
Watson, P. A., Arora, V. K., and Sawyers, C. L. (2015). Emerging mechanisms of resistance to androgen receptor inhibitors in prostate cancer. Nat Rev Cancer 15, 701-711.
Willard, et al., (2003). Site-specific quantitation of protein nitration using liquid chromatography/tandem mass spectrometry. Anal Chem 75, 2370-2376.
Zhu, et al. (2018). Loss of dihydrotestosterone-inactivation activity promotes prostate cancer castration resistance detectable by functional imaging. J Biol Chem.

Although only a number of exemplary embodiments have been described in detail, those skilled in the art will readily appreciate that many modifications are possible in the exemplary embodiments without materially departing from the novel teachings and advantages of this disclosure. Accordingly, all such modifications and alternative are intended to be included within the scope of the invention as defined in the following claims.

Those skilled in the art should also realize that such modifications and equivalent constructions or methods do not depart from the spirit and scope of the present disclosure, and that they may make various changes, substitutions, and alterations herein without departing from the spirit and scope of the present disclosure.

We claim:

1. A method of treating sex steroid dependent cancer comprising:
treating a subject having a sex steroid dependent cancer with a cytoplasmic tyrosine-protein kinase BMX (BMX) inhibitor.

2. The method claim 1, wherein said sex steroid dependent cancer is selected from the group consisting of: prostate cancer, breast cancer, ovarian cancer, and endometrial cancer.

3. The method of claim 1, wherein said BMX inhibitor comprises an anti-BMX monoclonal antibody or BMX binding portion thereof.

4. The method of claim 1, wherein said BMX inhibitor comprises zanubrutinib.

5. The method of claim 1, wherein said BMX inhibitor comprises acalabrutinib.

6. The method of claim 1, wherein said subject is 367T in the3βHSD1 protein.

7. The method of claim 1, further comprising conducting an assay on a sample from said subject to determine if said subject is 367T or 367N in their 3βHSD1 protein.

8. The method of claim 1, wherein said subject is heterozygous or homozygous for 1245C in the HSD3B1 gene.

9. The method of claim 1, further comprising conducting an assay on a sample from said subject to determine if said subject is heterozygous or homozygous for 1245C in the HSD3B1 gene.

10. The method of claim 1, wherein said subject is a human.

11. An in vitro composition comprising:
a) human sex steroid dependent cancer cells; and
b) a BMX inhibitor that is exogenous to said human sex steroid cancer cells.

12. The in vitro composition of claim 11, wherein said BMX inhibitor comprises an anti-BMX monoclonal antibody or BMX binding portion thereof.

13. The in vitro composition of claim 11, wherein said BMX inhibitor comprises zanubrutinib.

14. The in vitro composition of claim 11, wherein said BMX inhibitor comprises acalabrutinib.

15. The in vitro composition of claim 11, wherein said human sex steroid cancer cells comprise prostate cancer cells.

16. The in vitro composition of claim 1, wherein said prostate cancer cells are 367T in the3βHSD1 protein.

17. The in vitro composition of claim 1, wherein said sex steroid dependent cancer cells are heterozygous or homozygous for 1245C in the HSD3B1 gene.

18. The in vitro composition of claim 11, wherein said human sex steroid dependent cancer cells are breast cancer cells.

19. The in vitro composition of claim 11, wherein said human sex steroid dependent cancer cells are ovarian cancer cells.

20. The in vitro composition of claim 11, wherein said human sex steroid dependent cancer cells are endometrial cancer cells.


SEQUENCE LISTING (18 sequences)

SEQ ID NO: 1 - DNA, 21 bp, Homo sapiens
gatcacaatctgaacagttac

SEQ ID NO: 2 - DNA, 21 bp, Homo sapiens
gagtgctgataagaatgaata

SEQ ID NO: 3 - DNA, 21 bp, Homo sapiens
gcaatatgacagcaactcaaa

SEQ ID NO: 4 - DNA, 21 bp, Homo sapiens
cgtgcatacaaatgctgagaa

SEQ ID NO: 5 - DNA, 21 bp, Artificial sequence
  Synthetic
gcaatatgacagcaactcaaa

SEQ ID NO: 6 - DNA, 21 bp, Artificial sequence
  Synthetic
gatcacaatctgaacagttac

SEQ ID NO: 7 - DNA, 21 bp, Artificial sequence
  Synthetic
gaaggtttctgtcctaatcat

SEQ ID NO: 8 - DNA, 21 bp, Artificial sequence
  Synthetic
cgtttatactagcagaaaggc

SEQ ID NO: 9 - DNA, 22 bp, Artificial sequence
  Synthetic
ccatgtggtttgctgttaccaa

SEQ ID NO: 10 - DNA, 25 bp, Artificial sequence
  Synthetic
tcaaaacgaccctcaagttaaaaga

SEQ ID NO: 11 - DNA, 23 bp, Artificial sequence
  Synthetic
gcatgggatggggatgaagtaag

SEQ ID NO: 12 - DNA, 24 bp, Artificial sequence
  Synthetic
catcaaatctgagggttgtctgga

SEQ ID NO: 13 - DNA, 22 bp, Artificial sequence
  Synthetic
ccccctggtgaaccataataca

SEQ ID NO: 14 - DNA, 22 bp, Artificial sequence
  Synthetic
aaaaggccacctagctttttgc

SEQ ID NO: 15 - DNA, 20 bp, Artificial sequence
  Synthetic
ccatttgcaggatctgtctg

SEQ ID NO: 16 - DNA, 20 bp, Artificial sequence
  Synthetic
ggatgtgtcttggggagcaa

SEQ ID NO: 17 - DNA, 18 bp, Artificial sequence
  Synthetic
cgagggcacctggaaaac

SEQ ID NO: 18 - DNA, 19 bp, Artificial sequence
  Synthetic
cacattcccccggatatga